(19) United States
(12) Patent Application Publication
(10) Pub. No.: US 2015/0320398 A1
(43) Pub. Date: Nov. 12, 2015

(54) ULTRASONIC DIAGNOSTIC APPARATUS AND CONTROL METHOD

(71) Applicants: Kabushiki Kaisha Toshiba, Minato-ku (JP); Toshiba Medical Systems Corporation, Otawara-shi (JP)

(72) Inventors: Yasunori HONJO, Otawara (JP); Makoto HIRAMA, Otawara (JP); Akihiro KAKEE, Nasushiobara (JP); Kuramitsu NISHIHARA, Otawara (JP)

(73) Assignees: Kabushiki Kaisha Toshiba, Minato-ku (JP); Toshiba Medical Systems Corporation, Otawara-shi (JP)

(21) Appl. No.: 14/706,446

(22) Filed: May 7, 2015

(30) Foreign Application Priority Data
May 8, 2014 (JP) 2014-097145

(51) Int. Cl. A61B 8/08; A61B 8/00; A61B 8/14

(52) U.S. Cl. CPC A61B 8/5207; A61B 8/145; A61B 8/54; A61B 8/4444

(57) ABSTRACT

An ultrasonic diagnostic apparatus includes reception circuitry, signal processing circuitry, and generating circuitry. The reception circuitry outputs a plurality of reception signals corresponding to respective reception scanning lines for each transmission and reception of an ultrasonic wave by an ultrasonic probe. The signal processing circuitry executes a weighting process and a phase correction process according to the position of each reception scanning line on at least one of the reception signals and a plurality of signals based on the reception signals, generates the processed signals for each reception scanning line, and outputs a plurality of composite signals using the processed signals generated based on the transmission and reception of the ultrasonic waves before and after changing the sound field of the transmitted ultrasonic wave, and before and after changing the position of the reception scanning lines. The generating circuitry generates a piece of image data based on the composite signals.

### CROSS-REFERENCE TO RELATED APPLICATIONS

This application is based upon and claims the benefit of priority from Japanese Patent Application No. 2014-097145, filed on May 8, 2014, the entire contents of all of which are incorporated herein by reference.

### FIELD

An embodiment described herein relates generally to an ultrasonic diagnostic apparatus and a control method.

### BACKGROUND

Conventionally, various types of imaging methods have been adopted for ultrasonic diagnostic apparatuses depending on different purposes. For example, a parallel and simultaneous reception method is adopted for some ultrasonic diagnostic apparatuses to increase the frame rate (the time resolution). The parallel and simultaneous reception is a technology to increase the frame rate by setting a plurality of reception scanning lines in the sound field of a transmission beam and simultaneously receiving ultrasonic wave signals (reflected wave signals) from each reception scanning line. The conventional technology has been known as an applied technology of the parallel and simultaneous reception. With this applied technology, the reception signals on a reception scanning line are obtained for a plurality of times by changing the transmission scanning lines while overlapping some of the transmission scanning lines with the neighbor transmission beam, and the reception signals are added and composed to increase the signal-to-noise ratio.

A transmission wave front composition method is adopted for some ultrasonic diagnostic apparatuses to form a transmission beam and a reception beam having the uniform width in the depth direction in order to obtain images with higher spatial resolution. The transmission wave front composition is a technology to transmit a transmission beam focused in a certain depth on the transmission scanning lines, obtain the reception signals on a reception scanning line (on an observation point) for a plurality of times, then correct the reception signals using the delay amount resulting from the difference in the propagation distance of the transmission wave front (and the reception wave front), and finally compose the signals.

A multi-stage focus method is adopted for some ultrasonic diagnostic apparatuses to transmit a transmission beam for a plurality of times while changing the position of the transmission focal point on a scanning line in order to obtain images with uniformly higher resolution in the depth direction. An applied technology of the multi-stage focus has been also known in which the position of the transmission focal point is changed while changing the position of the transmission beam. The typical multi-stage focus requires transmissions for a plurality of times on a scanning line. By contrast, if the above-described applied technology of the multi-stage focus is used together with the above-described applied technology of the parallel and simultaneous reception, the multi-stage focus is achieved without increasing the number of transmission times, thereby preventing the frame rate from being decreased.

In the parallel and simultaneous reception, unfortunately, the increased number of the parallel and simultaneous receptions for the purpose of increasing the frame rate generates stripes at intervals of the simultaneous reception, because the reception is made from the position deviated from the sound field of the transmission beam. In addition, in the applied technology of the parallel and simultaneous receptions, unfortunately, the decreased number of reception scanning lines to be overlapped as small as possible for the purpose of increasing the frame rate generates irregularities of addition resulting from the difference of the number of compositions, because the number of composite times differs for each reception scanning line.

Although the transmission wave front composition is combined with the typical parallel and simultaneous reception or the above-described applied technology of the parallel and simultaneous receptions, the stripes resulting from the number of simultaneous receptions are not eliminated, because the advantageous effect obtained through the transmission wave front composition, that is, the increased resolution is limitedly observed in the vicinity of the transmission focal point.

Furthermore, although the above-described applied technology of the multi-stage focus is combined with the above-described applied technology of the parallel and simultaneous receptions, the irregularities of addition resulting from the difference of the number of compositions occur if the number of overlaps is set such that the number of composite times differs for each reception scanning line.

### BRIEF DESCRIPTION OF THE DRAWINGS

FIG. 1 is a block diagram illustrating the configuration of an ultrasonic diagnostic apparatus according to the present embodiment;

FIG. 2 is a first diagram for explaining an issue of the conventional technologies;

FIG. 3 is a second diagram for explaining an issue of the conventional technologies;

FIG. 4 is a third diagram for explaining an issue of the conventional technologies;

FIG. 5 is a diagram illustrating a scan sequence according to the present embodiment;

FIGS. 6A to 6C are diagrams illustrating settings on a transmission aperture used for the scan sequence according to the present embodiment;

FIG. 7 is a first diagram illustrating a process a receiver according to the present embodiment executes;

FIGS. 8A and 8B are second diagrams illustrating a process the receiver according to the present embodiment executes;

FIG. 9 is a first diagram illustrating a process a transmission phasing unit according to the present embodiment executes;

FIG. 10 is a second diagram illustrating a process the transmission phasing unit according to the present embodiment executes;

FIG. 11 is a third diagram illustrating a process the transmission phasing unit according to the present embodiment executes;

FIG. 12 is a fourth diagram illustrating a process the transmission phasing unit according to the present embodiment executes;

FIG. 13 is a fifth diagram illustrating a process the transmission phasing unit according to the present embodiment executes;

FIG. 14 is a diagram for explaining a first modification of the present embodiment;

FIG. 15 is a diagram for explaining a second modification of the present embodiment;

FIG. 16 is a block diagram illustrating the configuration of an ultrasonic diagnostic apparatus according to other embodiments; and

FIG. 17 is a flowchart of a processing procedure of the ultrasonic diagnostic apparatus according to other embodiments.

### DETAILED DESCRIPTION

An ultrasonic diagnostic apparatus according to the embodiment includes reception circuitry, signal processing circuitry, image generating circuitry. The reception circuitry outputs a plurality of reception signals corresponding to respective reception scanning lines for each transmission and reception of an ultrasonic wave by an ultrasonic probe. The signal processing circuitry executes a weighting process and a phase correction process according to the position of the reception scanning line on at least one of the reception signals or a plurality of signals based on the reception signals, and generates the processed signals for each reception scanning line. The signal processing circuitry outputs a plurality of composite signals using the processed signals generated based on the transmission and reception of the ultrasonic waves before and after changing the sound field of the transmitted ultrasonic wave, and before and after changing the position of the reception scanning lines. The image generating circuitry generates a piece of image data based on the composite signals output by the signal processing circuitry.

An exemplary embodiment of an ultrasonic diagnostic apparatus and a control method are described below in detail with reference to the accompanying drawings.

### Embodiment

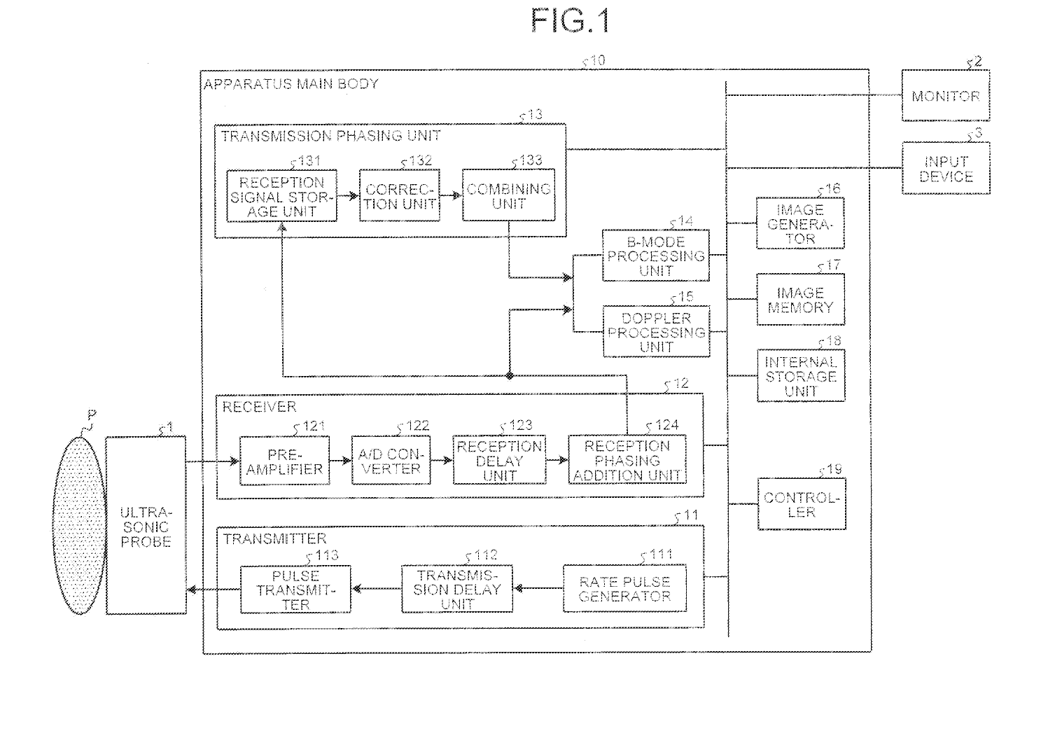

The configuration of an ultrasonic diagnostic apparatus according to the present embodiment will be described first. FIG. 1 is a block diagram illustrating the configuration of the ultrasonic diagnostic apparatus according to the present embodiment. As illustrated in FIG. 1, the ultrasonic diagnostic apparatus according to the present embodiment includes an ultrasonic probe 1, a monitor 2, an input device 3, and an apparatus main body 10.

The ultrasonic probe 1 includes, for example, a plurality of elements of a piezoelectric transducer and the elements generate ultrasonic waves based on driving signals provided by a later-described transmitter 11 included in the apparatus main body 10. The ultrasonic probe 1 receives a reflected wave from a subject P and converts it into electric signals. The ultrasonic probe 1 includes, for example, a matching layer provided on the piezoelectric transducer elements and a bucking material preventing propagation of ultrasonic waves from the piezoelectric transducer elements backward. The ultrasonic probe 1 is detachably coupled to the apparatus main body 10.

When the ultrasonic probe 1 transmits an ultrasonic wave to the subject P, the ultrasonic wave thus transmitted is reflected subsequently on a discontinuity surface of acoustic impedance in inner tissues of the subject P, and received by the elements included in the ultrasonic probe 1 as reflected wave signals. The amplitude of the reflected wave signals depends on a difference of the acoustic impedance on the discontinuity surface where the ultrasonic waves are reflected. If a transmitted ultrasonic pulse is reflected on a surface of a moving bloodstream or a moving cardiac wall, the reflected wave signals receive frequency shift due to the Doppler effect. The extent of the shift depends on a velocity component of a moving object in a transmitting direction of the ultrasonic wave.

The ultrasonic probe 1 is provided so as to be detachably coupled to the apparatus main body 10. If the subject P is two-dimensionally scanned (two-dimensional scanning), an operator couples a 1-D array probe, for example, as the ultrasonic probe 1 to the apparatus main body 10. The 1-D array probe has a plurality of piezoelectric transducer elements therein aligned in a row. Examples of the 1-D array probe include a linear ultrasonic probe, a convex ultrasonic probe, and a sector ultrasonic probe. If the subject P is three-dimensionally scanned (three-dimensional scanning), the operator couples a mechanical 4-D probe or a 2-D array probe, for example, as the ultrasonic probe 1 to the apparatus main body 10. The mechanical 4-D probe is capable of two-dimensional scanning by using a plurality of piezoelectric transducer elements aligned in a row in the same manner as the 1-D array probe. The mechanical 4-D probe is also capable of three-dimensional scanning by swinging the piezoelectric transducer elements at a certain angle (a swing angle). The 2-D array probe is capable of three-dimensional scanning by using a plurality of piezoelectric transducer elements aligned in a matrix. The 2-D array probe is also capable of two-dimensional scanning by converging and transmitting ultrasonic waves. The following describes an example in which the 1-D array probe is coupled to the apparatus main body 10.

The input device 3 has an input device such as a mouse, a keyboard, a button, a panel switch, a touch command screen, a foot switch, a trackball, and a joy stick. The input device 3 receives various types of setting demands from an operator of the ultrasonic diagnostic apparatus and then transfers the various types of setting demands thus received to the apparatus main body 10.

The monitor 2 displays, for example, a graphical user interface (GUI) for enabling the operator of the ultrasonic diagnostic apparatus to input various types of setting demands using the input device 3, or displays ultrasonic image data generated in the apparatus main body 10.

The apparatus main body 10 includes an apparatus that generates ultrasonic image data according to reflected wave signals received by the ultrasonic probe 1. The apparatus main body 10 illustrated in FIG. 1 includes an apparatus that can generate two-dimensional ultrasonic image data based on reflected wave data corresponding to the two-dimensional region of the subject P received by the ultrasonic probe 1. The apparatus main body 10 illustrated in FIG. 1 includes an apparatus that can generate three-dimensional ultrasonic image data based on reflected wave data corresponding to the three-dimensional region of the subject P received by the ultrasonic probe 1. As illustrated in FIG. 1, the apparatus main body 10 includes a transmitter 11, a receiver 12, a transmission phasing unit 13, a B-mode processing unit 14, a Doppler processing unit 15, an image generator 16, an image memory 17, an internal storage unit 18, and a controller 19.

The transmitter 11 transmits an ultrasonic wave from the ultrasonic probe 1. As illustrated in FIG. 1, the transmitter 11 includes a rate pulse generator 111, a transmission delay unit 112, and a pulse transmitter 113. The transmitter 11 provides driving signals to the ultrasonic probe 1. The rate pulse generator 111 repeatedly generates a rate pulse at a certain pulse repetition frequency (PRF) to form a transmission ultrasonic wave (a transmission beam). The rate pulses pass through the transmission delay unit 112, thereby including different transmission delay times and apply voltages to the pulse transmitter 113. The transmission delay unit 112, for example, converges ultrasonic waves generated from the ultrasonic probe 1 into a beam and provides a transmission delay time for each of the piezoelectric transducer elements necessary for determining transmitting directivity of the beam to the corresponding rate pulse generated by the rate pulse generator 111. The pulse transmitter 113 applies a driving signal (a drive pulse) to the ultrasonic probe 1 at a timing based on the rate pulse. The transmitter 11 controls the number of the transducer elements and position of the transducer elements (i.e., the transmission aperture) used for transmitting the ultrasonic waves, and transmission delay time based on the position of the transducer elements included in the transmission aperture, thereby providing the transmitting directivity. The transmission delay unit 112, for example, varies the transmission delay time to be provided to each rate pulse, thereby arbitrarily adjusting the transmitting direction from the surface of the piezoelectric transducer elements. It should be noted that the transmission delay time includes “0” when the delay time is applied.

The transmitter 11 according to the present embodiment is capable of executing, for example, the multi-stage focus in which the ultrasonic beam is transmitted a plurality of times on a common scanning line while changing the position (the depth) of the transmission focal point. If the transmitter 11 executes the multi-stage focus, the transmission delay unit 112 calculates the transmission delay time based on the depth of the transmission focal point and provides the calculated time to the pulse transmitter 113. The transmission delay time is usually calculated from the sound velocity value determined in advance as the average sound velocity of inner tissues of the subject P that is the imaged subject. A later-described controller 19 controls the transmitter 11 to execute the above-described different transmission controls by creating a wave front function for forming a desired transmission beam.

The drive pulse is transmitted from the pulse transmitter 113 thorough a cable to the piezoelectric transducer elements in the ultrasonic probe 1, and then converted from electrical signals to mechanical vibrations in the piezoelectric transducer elements. The ultrasonic waves generated from the mechanical vibrations are transmitted to inside of the patient's body. The ultrasonic waves having different transmission delay times for each of the piezoelectric transducer elements are converged and propagate in a given direction.

The transmitter 11 has the function of instantly changing a transmission frequency, a transmission driving voltage, and the like under the instruction of the controller 19 described later, in order to execute a certain scan sequence. Changing a transmission driving voltage, in particular, is achieved by a linear amplifier outgoing circuit that can instantly switch the voltage values, or a mechanism of electrically switching a plurality of power units.

The reflected wave of the ultrasonic waves transmitted by the ultrasonic probe 1 reaches the piezoelectric transducer elements inside of the ultrasonic probe 1. Subsequently, the reflected wave is converted from the mechanical vibration into electric signals (reflected wave signals) in the piezoelectric transducer elements and then input to the receiver 12. As illustrated in FIG. 1, the receiver 12 includes a preamplifier 121, an analog to digital (A/D) converter 122, a reception delay unit 123, and a reception phasing addition unit 124. The receiver 12 executes various types of processes on the reflected wave signals received by the ultrasonic probe 1 to generate reception signals (reflected wave data).

The preamplifier 121 amplifies the reflected wave signals for each channel and executes gain control on the signals. The A/D converter 122 converts the reflected wave signals that have been gain-corrected, from analog to digital. The signals output from the A/D converter 122 are, for example, IQ signals (complex signals) generated by converting the reflected wave signals that have been gain-corrected, into in-phase signals (I signals) and quadrature-phase signals (Q signals) in the baseband through the quadrature detection process or the Hilbert transformation process.

The reception delay unit 123 applies the reception delay (the reception delay time) necessary for determining reception directivity to the digital signals output by the A/D converter 122. Specifically, the reception delay unit 123 provides the reception delay times to the digital signals based on the distribution of the reception delay times for each reception focus calculated from the sound velocity value determined in advance as the average sound velocity of inner tissues of the subject P that is the imaged subject.

The reception phasing addition unit 124 adds the digital signals to which the reception delay times calculated from the average sound velocity are applied, to each other, thereby generating phased and added reception signals (a piece of reflected wave data). The addition process executed by the reception phasing addition unit 124 emphasizes the reflection component from the direction corresponding to the reception directivity of the reflected wave signals. That is, the reception delay unit 123 and the reception phasing addition unit 124 illustrated in FIG. 1 are processors for executing the delay and sum (DAS) method through the reception delay based on the average sound velocity, for example. The reception phasing addition unit 124 performs reception apodization. That is, the reception phasing addition unit 124 assigns weights to the signals (the input signals to which the reception delay times have been applied) from a sample point received by the elements of the reception aperture using the aperture function (the apodization function), and then executes the addition process on the weighted signals.

The receiver 12 according to the present embodiment is capable of executing parallel and simultaneous reception. The parallel and simultaneous reception is a technology to increase the frame rate (the time resolution) by setting a plurality of reception scanning lines in the sound field of a transmission beam and simultaneously receiving the ultrasonic wave signals (the reflected wave signals) from each reception scanning line. If the parallel and simultaneous reception is executed, the reception delay unit 123 and the reception phasing addition unit 124 execute a phasing addition process (a reception phasing addition process) using the reception delay times based on the position of the reception scanning lines. This will be described in detail later.

If the subject P is two-dimensionally scanned, the transmitter 11 transmits an ultrasonic beam for scanning the two-dimensional region of the subject P from the ultrasonic probe 1. The receiver 12 then generates two-dimensional reflected wave data from the two-dimensional reflected wave signals received by the ultrasonic probe 1. If the subject P is three-dimensionally scanned, the transmitter 11 transmits an ultrasonic beam for scanning the three-dimensional region of the subject P from the ultrasonic probe 1. The receiver 12 then generates three-dimensional reflected wave data from the three-dimensional reflected wave signals received by the ultrasonic probe 1.

The reflected wave data (IQ signals, that is, reception signals) output by the reception phasing addition unit 124 is input to at least one of the B-mode processing unit 14 and the Doppler processing unit 15, directly or through the transmission phasing unit 13. The reception signals output by the reception phasing addition unit 124 are output to the transmission phasing unit 13 if the scan sequence according to the present embodiment is executed. As illustrated in FIG. 1, the transmission phasing unit 13 includes a reception signal storage unit 131, a correction unit 132, and a combining unit 133. The transmission phasing unit 13 is a processor for executing a transmission phasing process. The process executed by the transmission phasing unit 13 will be described in detail later in addition to the scan sequence according to the present embodiment.

The B-mode processing unit 14 performs, for example, logarithm amplification, envelope detection processing, and logarithmic compression on the reflected wave data output by the reception phasing addition unit 124 or the transmission phasing unit 13, thereby generating data whose signal intensity (amplitude strength) for each sample point is represented by a degree of brightness (i.e., B-mode data).

The Doppler processing unit 15 performs frequency analysis of the reflected wave data output by the reception phasing addition unit 124 or the transmission phasing unit 13, thereby generating data resulting from extracting the moving information on a moving object (e.g., bloodstream, a tissue, and a contrast agent) based on the Doppler effect (i.e., Doppler data). Specifically, the Doppler processing unit 15 generates Doppler data resulting from extracting an average speed, dispersion, and power on many points as moving information on the moving object.

The B-mode processing unit 14 and the Doppler processing unit 15 are capable of processing both two-dimensional reflected wave data and three-dimensional reflected wave data.

The image generator 16 generates the ultrasonic image data from the data generated by the B-mode processing unit 14 and the Doppler processing unit 15. The image generator 16 generally converts signal columns of scanning lines in ultrasonic scanning into signal columns of scanning lines in video format that is typical in a television (scan conversion), thereby generating ultrasonic image data for display. Specifically, the image generator 16 performs coordinates transformation according to the scan mode of ultrasonic waves by the ultrasonic probe 1, thereby generating the ultrasonic image data for display. The image generator 16 performs various types of image processing in addition to the scan conversion. For example, the image generator 16 performs image processing using a plurality of image frames after the scan conversion to regenerate an image of averaged brightness values (smoothing processing). For another example, the image generator 16 performs image processing using a differential filter on images (edge enhancement processing). In addition, the image generator 16 superimposes character information of various parameters, a scale, a body mark, for example, onto ultrasonic image data.

The B-mode data and the Doppler data are the ultrasonic image data before the scan conversion. The image generator 16 generates the ultrasonic image data after the scan conversion to be displayed. The B-mode data and Doppler data are also called raw data.

For another example, the image generator 16 performs coordinates transformation on the three-dimensional B-mode data generated by the B-mode processing unit 14, thereby generating three-dimensional B-mode image data. Furthermore, the image generator 16 performs coordinates transformation on the three-dimensional Doppler data generated by the Doppler processing unit 15, thereby generating three-dimensional Doppler image data. That is, the image generator 16 generates “three-dimensional B-mode image data and three-dimensional Doppler image data” as “three-dimensional ultrasonic image data (volume data)”. Subsequently, the image generator 16 performs various types of rendering process on the volume data in order to generate various types of two-dimensional image data for displaying the volume data on the monitor 2.

The image memory 17 is a memory that stores therein the image data generated by the image generator 16. The image memory 17 is also capable of storing therein the data generated by the B-mode processing unit 14 or the Doppler processing unit 15. The B-mode data and the Doppler data stored in the image memory 17 can be retrieved by an operator after a diagnosis, for example, which serve as ultrasonic image data for display through the image generator 16. The image memory 17 is also capable of storing the data output by the receiver 12 or the data output by the transmission phasing unit 13.

The internal storage unit 18 stores therein a control program for performing ultrasonic transmission/reception, image processing, or display processing, and various types of data such as diagnostic information (e.g., a patient ID, doctor's findings), diagnostic protocols, and various body marks. The internal storage unit 18 is used for storing the data stored in the image memory 17, as necessary.

The controller 19 controls processing of the ultrasonic diagnostic apparatus totally. Specifically, the controller 19 controls processing of the transmitter 11, the receiver 12, the transmission phasing unit 13, the B-mode processing unit 14, the Doppler processing unit 15, and the image generator 16 according to various types of setting demands input from the operator through the input device 3 or various types of control programs and various types of data read from the internal storage unit 18. The controller 19 controls the monitor 2 to display the ultrasonic image data for display stored in the image memory 17.

The entire configuration of the ultrasonic diagnostic apparatus according to the present embodiment has been described. With the configuration, the ultrasonic diagnostic apparatus according to the present embodiment generates and displays ultrasonic image data (e.g., B-mode image data). As described above, the ultrasonic diagnostic apparatus illustrated in FIG. 1 is capable of executing the parallel and simultaneous reception to improve the frame rate (the time resolution). As described above, the ultrasonic diagnostic apparatus illustrated in FIG. 1 is also capable of executing the multi-stage focus to generate ultrasonic image data with uniformly higher resolution in the depth direction.

The ultrasonic diagnostic apparatus illustrated in FIG. 1 is also capable of executing transmission wave front composition in which the receiver 12 and some functions of the transmission phasing unit 13 are used to form a transmission beam and a reception beam each having the uniform width in the depth direction. The transmission wave front composition is a technology to transmit a transmission beam focused in a certain depth on the respective transmission scanning lines, obtain the reception signals on a reception scanning line (on an observation point), then correct the reception signals using the delay amount resulting from the difference in the propagation distance of the transmission wave front (and the reception wave front), and finally compose the signals.

The conventional technology has been known as an applied technology of the parallel and simultaneous receptions. With this technology, the reception signals on a reception scanning line are obtained a plurality of times by changing the transmission scanning lines while overlapping the transmission scanning lines with a neighbor transmission beam, and the reception signals are added and composed to increase the signal-to-noise ratio. The number of the reception signals to be added and combined with each other are defined as, for example, “the number of overlaps”. If the setting “the number of simultaneous receptions: 4, the number of overlaps: 2” is made, the controller 19 sets four reception scanning lines in the sound field of the transmission beam and aligns the position of the transmission aperture such that two reception scanning lines out of the four reception scanning lines are overlapped with four reception scanning lines set on a transmission beam next to the transmission beam.

The reception signals on the two reception scanning lines overlapped are added and combined with each other, thereby increasing the signal-to-noise ratio. The frame rate in the parallel and simultaneous reception under the setting “the number of simultaneous receptions: 4, the number of overlaps: 0” is four times as large as the frame rate in usual scanning without the parallel and simultaneous reception. By contrast, the frame rate in the parallel and simultaneous reception under the setting “the number of simultaneous receptions: 4, the number of overlaps: 2” is half the frame rate in the parallel and simultaneous reception under the setting “the number of simultaneous receptions: 4, the number of overlaps: 0” and is twice as large as the frame rate in usual scanning without the parallel and simultaneous reception.

Another conventional technology has been known in which the transmission wave front composition is executed together with the parallel and simultaneous reception. An applied technology of the multi-stage focus has been also known in which the position of the transmission focal point is changed while changing the transmission beam to increase the frame rate, because “N times” of transmission/reception is performed on a scanning line where “N” represents the number of the transmission focal points in the multi-stage focus. In addition, another technology has been also known in which the parallel and simultaneous reception of overlapping, adding, and composing the reception scanning lines is used together with the above-described applied technology of the multi-stage focus.

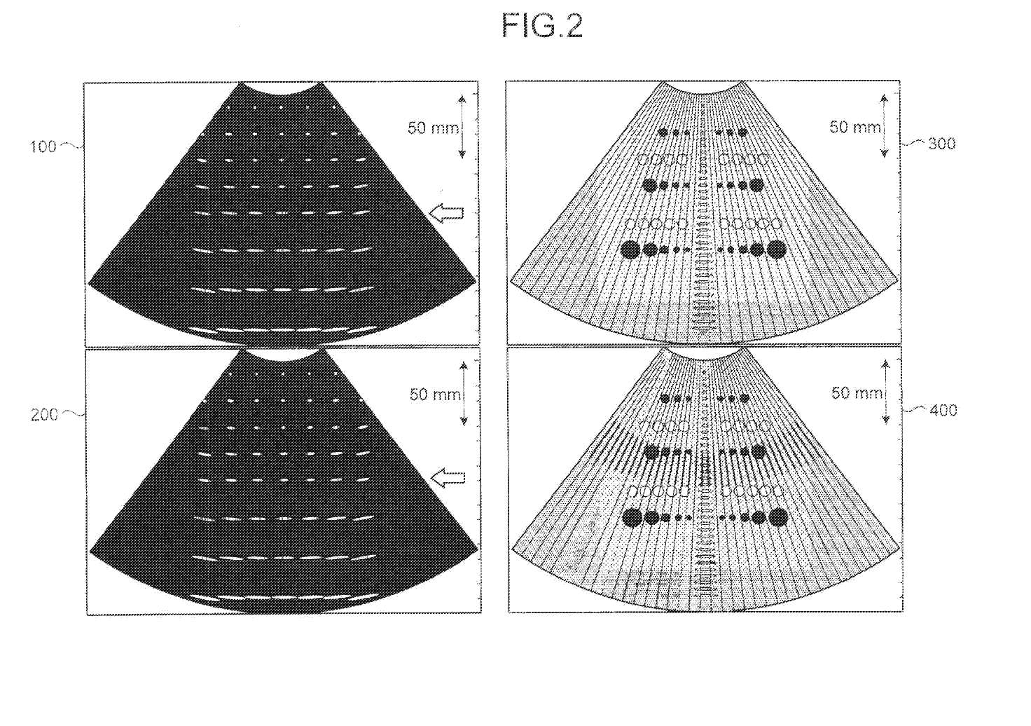

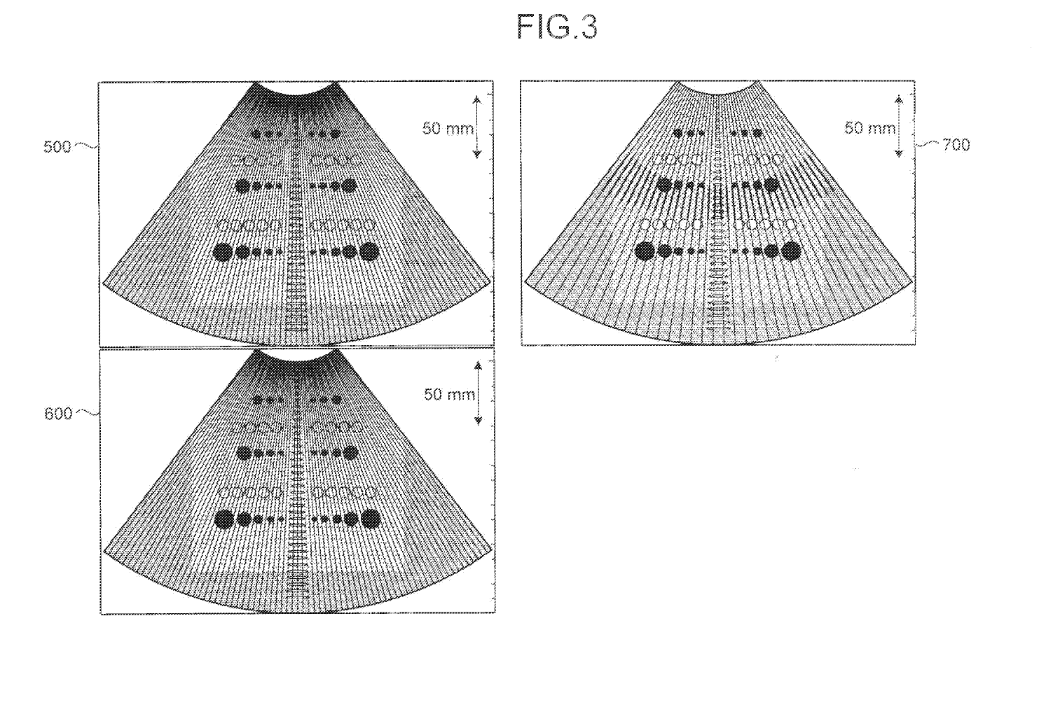

The above-described different conventional technologies, however, may have difficulties in increasing both the time resolution and the spatial resolution. This will be described with reference to FIGS. 2 to 4. FIGS. 2 to 4 are diagrams for explaining an issue of the conventional technologies. FIG. 2 schematically illustrates the result of phantom simulation in which the parallel and simultaneous reception is executed under the setting “the number of simultaneous receptions: 8, the number of overlaps: 0” to increase the frame rate to eight times as large as the frame rate in usual scanning. FIG. 3 schematically illustrates the result of the parallel and simultaneous reception executed under the setting “the number of simultaneous receptions: 8, the number of overlaps: 2” to increase the signal-to-noise ratio and increase the frame rate to six times as large as the frame rate in usual scanning.

An image data 100 and an image data 300 illustrated in FIG. 2 are pieces of B-mode image data obtained through the parallel and simultaneous reception executed under the setting “the number of simultaneous receptions: 8, the number of overlaps: 0”. An image data 200 and the image data 400 illustrated in FIG. 2 are pieces of B-mode image data obtained through the parallel and simultaneous reception executed under the setting “the number of simultaneous receptions: 8, the number of overlaps: 0” and by applying the phase correction executed in the transmission wave front composition. The position of the transmission focal point in the image data 100, 200, 300, and 400 is each set to “80 mm”. The image data 100 and 200 are analyzed under the common conditions, and the image data 300 and 400 are analyzed under the common conditions.

The image data 200 schematically illustrates the increased azimuth resolution of the transmission focal point due to the phase correction in comparison with the image data 100 (refer to the arrows in FIG. 2). The advantageous effect obtained through the transmission wave front composition, that is, the increased resolution, however, is limitedly observed in the vicinity of the transmission focal point, as schematically illustrated in the image data 200.

In the parallel and simultaneous reception, unfortunately, the increased number of the parallel and simultaneous receptions for the purpose of increasing the frame rate generates stripes at intervals of the simultaneous reception as schematically illustrated in the image data 300, because the reception is made from the position deviated from the sound field of the transmission beam. Although not illustrated in FIG. 2, for drawing convenience, the image data 100 and 200 have actually therein such stripes at intervals of the simultaneous reception. The image data 400, which has been subjected to the transmission phase correction of the transmission wave front composition, still schematically illustrates such stripes at intervals of the simultaneous reception in the same manner as the image data 300. This is because, as described above, the advantageous effect obtained through the transmission wave front composition, that is, the increased resolution is limitedly observed in the vicinity of the transmission focal point.

An image data 500, 600, and 700 illustrated in FIG. 3 are analyzed under the same conditions in the phantom used for the image data 300 and 400. Specifically, the image data 500 in FIG. 3 schematically illustrates a piece of B-mode image data obtained through the parallel and simultaneous reception under the setting “the number of simultaneous receptions: 8, the number of overlaps: 2”. The image data 700 in FIG. 3 schematically illustrates a piece of B-mode image data obtained through the parallel and simultaneous reception under the setting “the number of simultaneous receptions: 8, the number of overlaps: 2” and by applying the transmission wave front. The position of the transmission focal point in the image data 500 and 700 is each set to “80 mm”. The image data 600 in FIG. 3 schematically illustrates a piece of B-mode image data obtained through the parallel and simultaneous reception executed under the setting “the number of simultaneous receptions: 8, the number of overlaps: 2” using a transmission beam with the position of the transmission focal point “40 mm” and a transmission beam with the position of the transmission focal point “80 mm” and by simply combining the two kinds of the reception signals, or, the two kinds of the B-mode image data.

The image data 500 adopts an unusual setting of the number of overlaps “2” to increase the frame rate. This means that regions having the composed number “2” and regions having the composed number “1” (no composition) alternatively appear in the azimuth direction. Accordingly, the image data 500 schematically illustrates increased irregularities of addition resulting from the difference of the number of compositions in comparison with the image data 300. The image data 500 also schematically illustrates the stripes at intervals of the simultaneous reception in the same manner as the image data 300. The image data 600 still schematically illustrates the remaining irregularities of addition found in the image data 500 because the image data 600 is the combined result of different pieces of data each having different position of the transmission focal point. The image data 700 also schematically illustrates stripes and irregularities of addition found in the image data 500 because the advantageous effect obtained through the transmission wave front composition is limited.

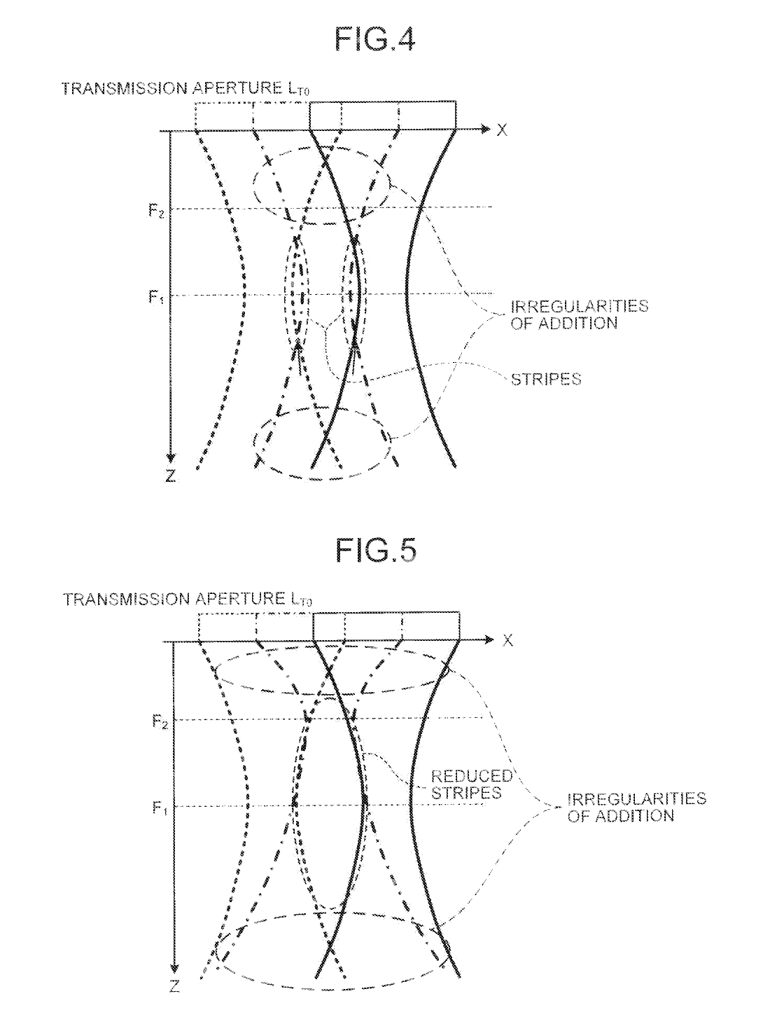

The following describes the above-described stripes and irregularities of addition in greater detail with reference to FIG. 4. FIG. 4 illustrates that the position of the transmission focal point of the transmission beam is set to “F₁” and the parallel and simultaneous reception is executed with overlaps while shifting the position of “the transmission aperture L_{T0}” between the transmission rates. FIG. 4 illustrates three lines of transmission beam. As represented with the vertical ellipses illustrated in FIG. 4, the stripes at intervals of the simultaneous reception are generated around “F₁” along the depth direction. That is, the transmission beams are mostly focused at the depth “F₁”, so that the parallel and simultaneous reception generates the stripes at intervals of the simultaneous reception around the transmission focal point “F₁”. As represented with the horizontal ellipses illustrated in FIG. 4, the irregularities of addition are generated due to overlap.

As described above, the conventional technologies may have difficulties in increasing both the time resolution and the spatial resolution. The ultrasonic diagnostic apparatus according to the present embodiment thus executes the following processes to increase both the time resolution and the spatial resolution.

Firstly, the transmitter 11 changes the transmission focal point position of the transmission ultrasonic wave transmitted from the ultrasonic probe 1 to any one of a plurality of transmission focal points for each transmission ultrasonic wave. In other words, the controller 19 controls the transmitter 11 to set a plurality of transmission focal points and transmit the transmission beam with the changed transmission focal point position for each transmission rate from the ultrasonic probe 1.

Specifically, the transmitter 11 changes the transmission focal point position on the respective transmission scanning lines while changing the transmission scanning line position for each transmission ultrasonic wave. For example, the transmitter 11 transmits the transmission beam with the changed transmission focal point position on the respective transmission scanning lines while changing the transmission scanning line position for each transmission rate. FIG. 5 is a diagram illustrating a scan sequence according to the present embodiment.

The scan sequence in FIG. 5 illustrates that if the depth “F₁” and the depth “F₂” are set as a plurality of transmission focal points, the parallel and simultaneous reception is executed with overlaps while shifting the position of “the transmission aperture L_{T0}” between the transmission rates using the transmission beam with the transmission focal point position “F₁” and the transmission beam with the transmission focal point position “F₂”. That is, the transmitter 11 alternatively sets the depth of the transmission focal point to “F₁” and “F₂” while walking the transmission beam in an example of the scan sequence according to the present embodiment. With the scan sequence illustrated in FIG. 5, the depth where the transmission beam is mostly focused is changed whereby the depth where the stripes at intervals of the simultaneous reception are likely to occur is changed. Therefore, some stripes are prevented from being generated in comparison with the conventional scan sequence illustrated in FIG. 4.

As a modification of the above-described scan sequence, the transmitter 11 according to the present embodiment may change the transmission focal point position on a transmission scanning line for each transmission ultrasonic wave. As a modification of the above-described scan sequence, the transmitter 11 may transmit the transmission beam with the changed transmission focal point position on a transmission scanning line for each transmission rate. In the modification, for example, the transmitter 11 transmits the transmission beam with the transmission focal point position “F₁” once and the transmission beam with the transmission focal point position “F₂” once, without changing the transmission scanning line position. Subsequently, the transmitter 11 shifts the position of “the transmission aperture L_{T0}”, and transmits the transmission beam with the transmission focal point position “F₁” and the transmission beam with the transmission focal point position “F₂”. In the modification, the depth where the transmission beam is mostly focused is also changed whereby the depth where the stripes at intervals of the simultaneous reception are likely to occur is changed. Therefore, some stripes are prevented from being generated.

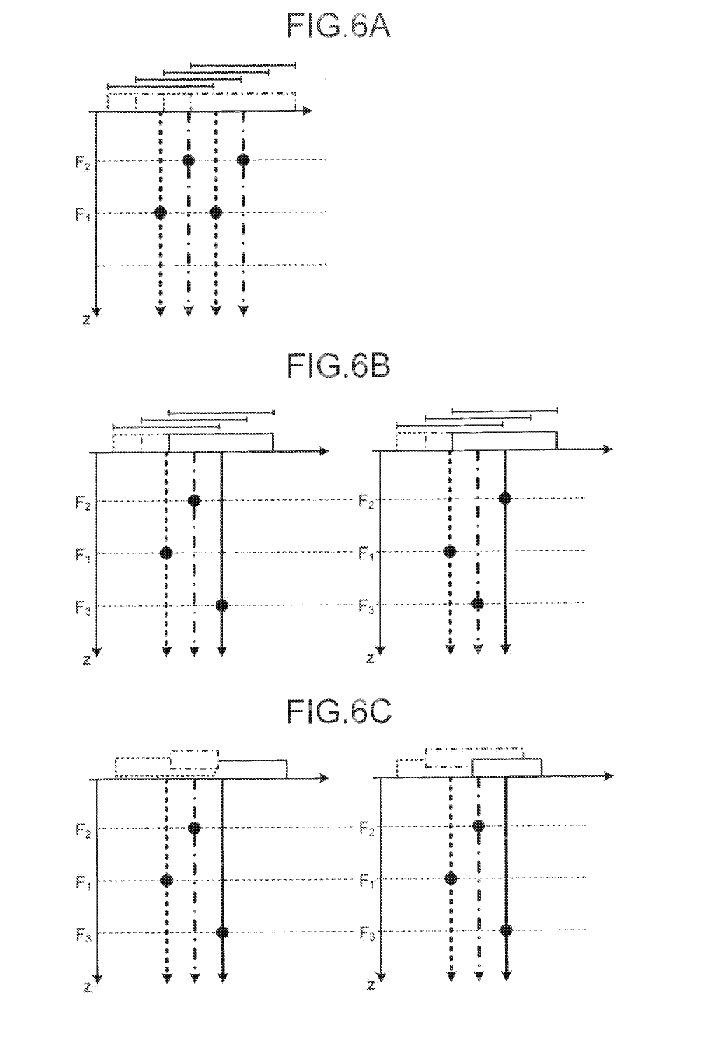

The aperture width of the transmitter 11 when transmitting the transmission ultrasonic wave (the transmission beam) for a plurality of transmission focal points may be set to be any desirable aperture width, a fixed aperture width, or an aperture width depending on the transmission focal point position. FIGS. 6A to 6C are diagrams illustrating the settings on the transmission aperture used for the scan sequence according to the present embodiment. FIGS. 6A to 6C illustrate the scan sequence that changes the depth of the transmission focal point while walking the transmission beam.

FIG. 6A illustrates the scan sequence that have two transmission focal points “F₁” and “F₂” in the same manner as FIG. 5 and switches the transmission focal points in the order of “F₁, F₂” while walking the raster. The left-hand diagram on FIG. 6B illustrates the scan sequence that have three transmission focal points “F₁”, “F₂”, and “F₃” and switches the transmission focal points in the order of “F₁, F₂, F₃” while walking the raster. The right-hand diagram on FIG. 6B illustrates the scan sequence, as a modification of the scan sequence in the left-hand diagram on FIG. 6B, that switches the transmission focal points in the order of “F₁, F₃, F₂” while walking the raster. FIGS. 6A and 6B illustrate that the width of the transmission aperture used for the transmission beam for each transmission focal point is set to a fixed value.

By contrast, FIG. 6C illustrates a modification of the scan sequence in the left-hand diagram on FIG. 6B in regard to the setting on the transmission aperture. The left-hand diagram on FIG. 6C illustrates that the width of the transmission aperture is set depending on the depth of “F₁”, “F₂” and “F₃” (e.g., depending on the transmission F-number). In addition, the right-hand diagram on FIG. 6C illustrates that the width of the transmission aperture is arbitrarily set for “F₁”, “F₂” and “F₃”. It should be noted that an operator, for example, can arbitrarily change the number of the transmission focal points and the setting on the transmission aperture on the ultrasonic diagnostic apparatus according to the present embodiment.

So far described is the scan sequence that prevents the stripes (the vertical stripes) from being generated at intervals of the simultaneous reception in the parallel and simultaneous reception according to the present embodiment. In the process to compose the signals by overlapping the reception scanning lines during the parallel and simultaneous reception, however, the irregularities of addition resulting from the difference of the number of compositions still occur as illustrated in FIG. 5.

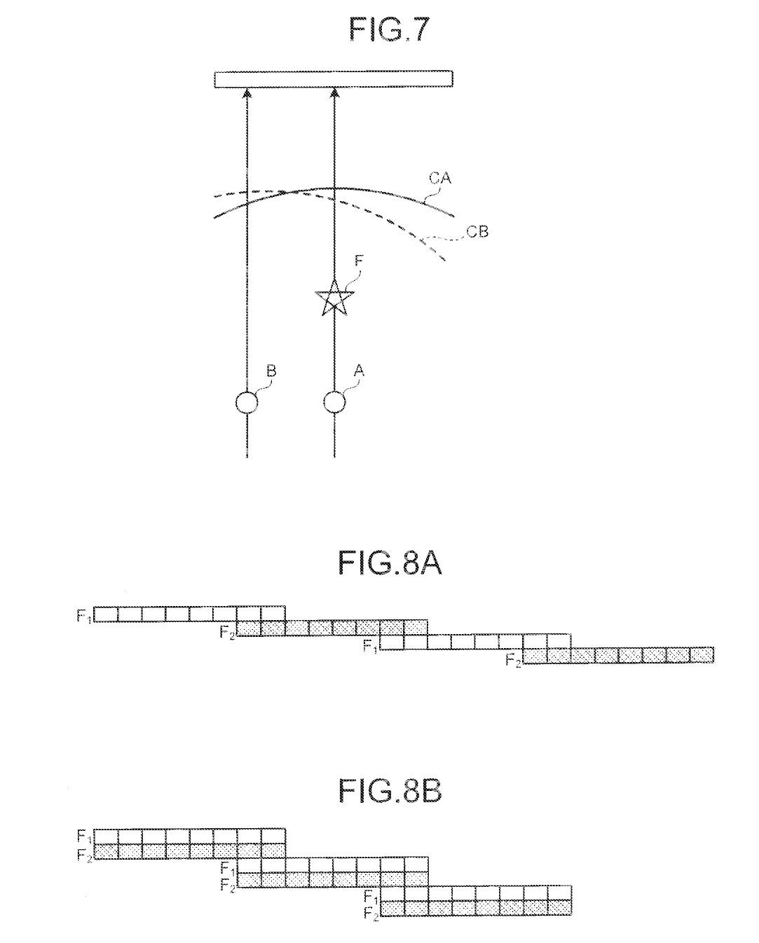

The ultrasonic diagnostic apparatus according to the present embodiment thus executes the following processes to eliminate the irregularities of addition. Firstly, the receiver 12 performs phasing addition using the reception delay time based on the position of the reception scanning lines for each reflected wave of the transmission ultrasonic wave. The receiver 12 then outputs a plurality of reception signals corresponding to the respective reception scanning lines through the phasing addition from the reflected wave signals received by the ultrasonic probe 1. For example, the controller 19 controls the receiver 12 to set a plurality of reception scanning lines on which the parallel and simultaneous reception is performed on transmission beams. In the present embodiment, the controller 19 controls the receiver 12 to set the reception scanning lines such that a part of the reception scanning lines within the sound field of a transmission beam overlaps with a part of the reception scanning lines within the sound field of the neighbor transmission beam. The receiver 12, for example, makes the setting “the number of simultaneous receptions: 8, the number of overlaps: 2”. Subsequently, the receiver 12 performs phasing addition using the reception delay time based on the position of the reception scanning lines. The receiver 12 then outputs a plurality of reception signals corresponding to the respective reception scanning lines through the phasing addition from the reflected wave signals received by the ultrasonic probe 1. The phasing addition using the reception delay time based on the position of the reception scanning lines is performed by the reception delay unit 123 and the reception phasing addition unit 124. FIGS. 7, 8A, and 8B are diagrams illustrating the processes the receiver according to the present embodiment executes.

The outlined white rectangle illustrated in FIG. 7 represents a reception aperture. The star F illustrated in FIG. 7 represents the transmission focal point position that is the center of the transmission beam. The point A illustrated in FIG. 7 represents a sample point on the reception scanning line on the same position as the transmission scanning line of a transmission beam, for example, out of the reception scanning lines simultaneously received. The point B illustrated in FIG. 7 represents a sample point on the reception scanning line apart from the transmission scanning line of the transmission beam out of the reception scanning lines simultaneously received.

The transmission beam focused at the position of the star F propagates as a spherical wave with the star F as the virtual sound source, for example. That is, the wave front from the star F reaches the point A, and it is reflected at the point A, then received by the elements of the reception aperture. When the receiver 12 outputs the reception signals (IQ signals) focused at the point A, therefore, the reception delay unit 123 calculates a reception delay curve CA illustrated in FIG. 7 based on the distance from the star F to the point A and the distance from the point A to the elements. The reception delay unit 123 then applies the delay to the signals and outputs the signals to the reception phasing addition unit 124.

The wave front from the star F reaches the point B, and it is reflected at the point B, then received by the elements of the reception aperture. When the receiver 12 outputs the reception signals (IQ signals) focused at the point B, therefore, the reception delay unit 123 calculates the reception delay curve CB illustrated in FIG. 7 based on the distance from the star F to the point B and the distance from the point B to the elements. The reception delay unit 123 then applies the delay to the signals and outputs the signals to the reception phasing addition unit 124. The same reception correction processes as described above are executed when the receiver 12 outputs the reception signals at the sample points on the reception scanning lines passing through the point A, the reception signals at the sample points on the reception scanning line passing through the point B, and the reception signals at the sample points on other reception scanning lines for the simultaneous reception.

Subsequently, the reception phasing addition unit 124 outputs the reception signals on the reception scanning lines to the transmission phasing unit 13. When the scan sequence illustrated in FIG. 5 is executed, for example, the reception phasing addition unit 124 outputs the reception signals on the eight reception scanning lines simultaneously received in the transmission beam having the transmission focal point “F₁”, as illustrated in FIG. 8A. Subsequently, the reception phasing addition unit 124 outputs the reception signals on the eight reception scanning lines simultaneously received in the transmission beam having the transmission focal point “F₂”, as illustrated in FIG. 8A. FIG. 8A illustrates that the two scanning lines on the right-end side out of the eight reception scanning lines obtained at “F₁” overlap the two scanning lines on the left-end side out of the eight reception scanning lines obtained at “F₂”.

When the scan sequence described as a modification is executed, the reception phasing addition unit 124 outputs, as illustrated in FIG. 8B, the reception signals on the eight reception scanning lines simultaneously received in the transmission beam having the transmission focal point “F₁” and the reception signals on the eight reception scanning lines simultaneously received in the transmission beam having the transmission focal point “F₂” on the identical transmission scanning lines. FIG. 8B illustrates that the respective two scanning lines on the right-end side out of the respective eight reception scanning lines obtained at “F₁” and “F₂” overlap the respective two scanning lines on the left-end side out of the respective eight reception scanning lines obtained at “F₁” and “F₂”.

The reception signals on the reception scanning lines for each transmission rate that are output by the reception phasing addition unit 124 are sequentially stored in the reception signal storage unit 131. After the reception signals for one frame is stored in the reception signal storage unit 131, for example, the correction unit 132 starts processing.

The correction unit 132 executes an amplitude weighting process and a phase correction process on the reception signals depending on the position of the reception scanning lines, and outputs a plurality of processed reception signals. In other words, the correction unit 132 executes an amplitude correction on the reception signals corresponding to the reception scanning lines, and a transmission delay correction on the transmission beam in the transmission rate from which the reception signals are obtained. The correction unit 132 then outputs a plurality of corrected reception signals, that is, a plurality of processed reception signals. Specifically, the correction unit 132 calculates the weight of amplitude used for the weighting process on the reception signals and the phase correction amount used for the phase correction process based on the transmission focal point position of the transmission ultrasonic wave from which the reception signals are obtained. It should be noted that, hereinafter, the “weighting process” may be referred to as the “amplitude correction”, and the “weight of amplitude used for the weighting process” may be referred to as an “amplitude correction amount”. It should also be noted that, hereinafter, the “phase correction process” may be referred to as a “transmission delay correction”, and the “phase correction amount used for the phase correction process” may be referred to as a “delay correction amount used for transmission delay correction”. The correction unit 132 calculates the weight of amplitude used for the weighting process and the phase correction amount used for the phase correction process on the reception signals based on the transmission focal point position of the transmission ultrasonic wave from which the reception signals are obtained (i.e., the transmission beam in the transmission rate from which the reception signals are obtained). The correction unit 132 calculates the delay correction amount (the phase correction amount) on the reception signals based on the relative distance differences in propagation paths for the transmission ultrasonic wave (the transmission beam) from which the reception signals are obtained to reach the respective reception scanning lines. The correction unit 132 executes, for example, the transmission delay correction on the reception signals based on the phases in the propagation paths of the transmission beam in the transmission rate from which the reception signals are obtained.

The correction unit 132 also calculates the weight of amplitude (the amplitude correction amount) on the reception signals based on the parameter in regard to the transmission ultrasonic wave (the transmission beam) from which the reception signals are obtained. The correction unit 132 uses, for example, the distance from the transmission ultrasonic wave to the reception scanning lines, as the parameter in regard to the transmission ultrasonic wave (the transmission beam).

Subsequently, the combining unit 133 combines a plurality of processed reception signals (a plurality of corrected reception signals) on a reception scanning line out of a plurality of corrected reception signals on the transmission ultrasonic waves (the transmission rates) that are output by the correction unit 132. Subsequently, the image generator 16 generates image data based on the signals that are output by the combining unit 133. Specifically, the image generator 16 generates B-mode image data from the B-mode data generated by the B-mode processing unit 14 based on the signals that are output by the combining unit 133. Subsequently, the controller 19 controls the monitor 2 to display the B-mode image data thereon.

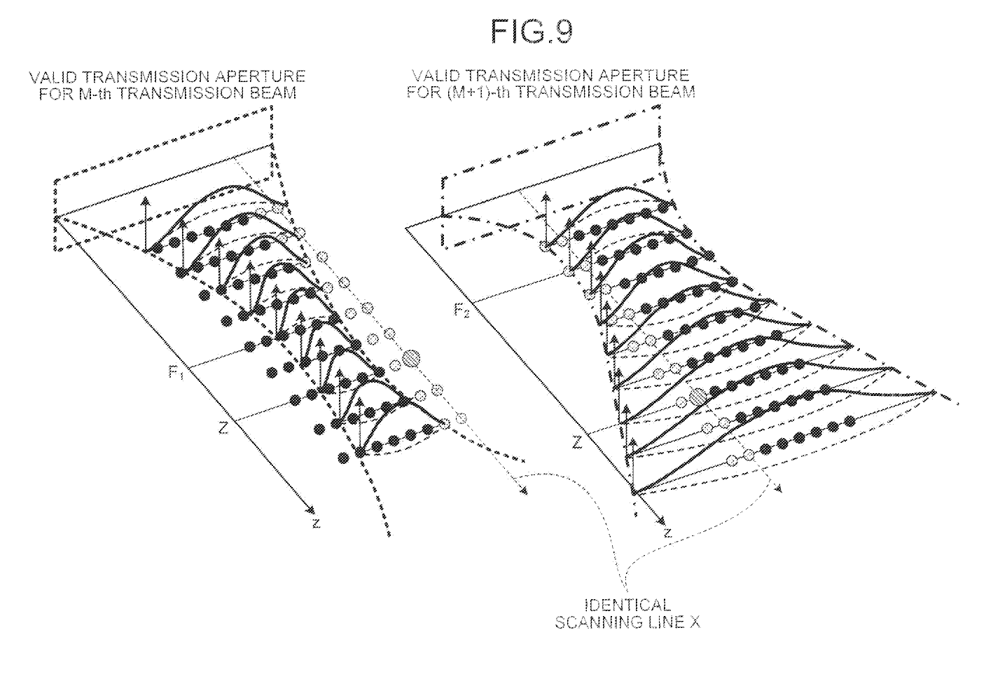

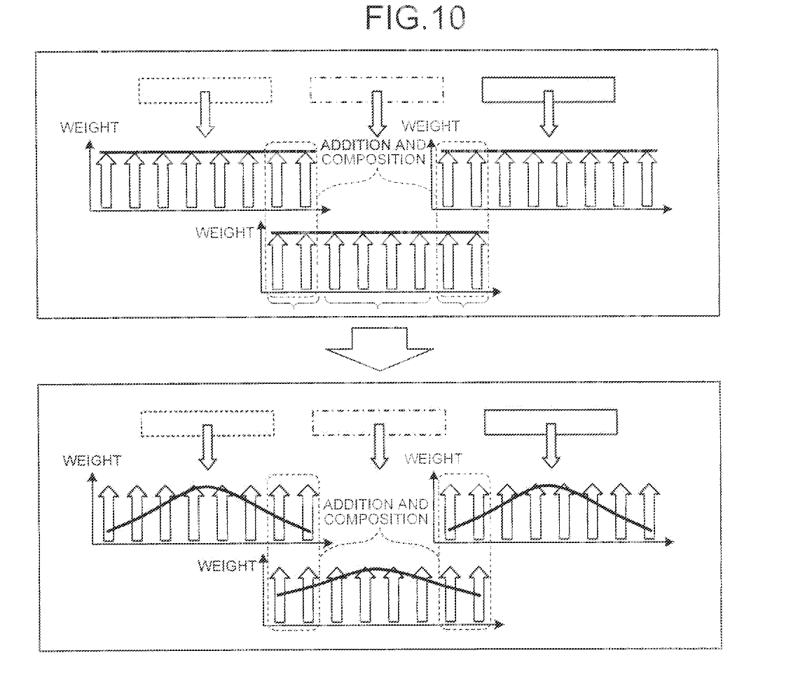

The following describes an example of processes executed by the correction unit 132 and the combining unit 133 when the scan sequence illustrated in FIG. 5 is executed in the parallel and simultaneous reception under the setting “the number of simultaneous receptions: 8, the number of overlaps: 2”, with reference to FIGS. 9 to 13. FIGS. 9 to 13 are diagrams illustrating the processes the transmission phasing unit according to the present embodiment executes.

Firstly, the weighting process of the amplitude will be described with reference to FIGS. 9 and 10. FIG. 9 illustrates the disposition of the transmission/reception beams when the number of simultaneous receptions is set to “8” in the “M-th” transmission beam and the number of overlaps with the “(M+1)-th” transmission beam transmitted at the position after walking is set to “2”. The hatched circles in FIG. 9 illustrate sample points on two overlapped reception scanning lines. The “curves” in FIG. 9 schematically illustrate amplitude correction values given by the correction unit 132 used for weights to the reception signals on the eight sample points at the same depth on the respective eight reception scanning lines in the transmission rates.

The “curves” illustrated in FIG. 9 represent amplitude correction values (weighting functions) calculated by the correction unit 132 using the distances from the center of the transmission beams (the transmission focal points) to the respective sample points as a parameter in regard to the transmission beam. The upper diagram on FIG. 10 illustrates that the transmission beams are transmitted from the three transmission apertures on different positions and the parallel and simultaneous reception is performed. Thus obtained respective eight reception signals are added and combined with each other with uniform weights (i.e., without any amplitude correction). If the reception signals on the respective two reception scanning lines at both ends are added and combined without any amplitude correction, irregularities of addition occur depending on presence of addition.

By contrast, the lower diagram on FIG. 10 illustrates that the weight of amplitude is assigned to the respective eight reception signals obtained in the transmission rates using the weighting function illustrated in FIG. 9. The weighted reception signals on the respective two reception scanning lines at both ends are then added and combined with each other, whereby some irregularities of addition are prevented from being generated.

The following describes a calculation method of the weighting function used for preventing such irregularities of addition from being generated with reference to some numerical expressions. For example, the number “m” of focal points are set at a certain depth “Z_{Fm}, m=0, 1, 2 . . . ” in a transmission beam. The weight based on the distance between the scanning line position “(x(n),z), n=0, 1, 2 . . . ” and the center position “(x₀(k),z)” of the transmission beam “Tₖ” are used and composition is executed in the transmission beam. Subsequently, if the amplitude distribution of the transmission waveform is the Gaussian distribution, the correction unit 132 calculates the weighting function “W_{Fm}(x(n),z;Tₘ)” through the following Expression (1).

$${W}_{{F}_{m}} ( x ( n ) , z ; {T}_{m} ) = exp [ - \frac{{( x ( n ) - {x}_{0} ( k ) )}^{2}}{{B}^{2} ( z ; {Z}_{{F}_{m}} )} ] \qquad ( 1 )$$

In Expression (1), “x(n)” represents the scanning line position at the simultaneous reception point “n” in the azimuth direction. In addition, “x₀(k)” represents the center position (the position of the transmission scanning line in the azimuth direction) of the transmission beam “Tₖ” on the transmission beam number “k”. Furthermore, “B (z;Z_{Fm})” represents the beam width at the depth “z” of the transmission beam “Tₖ” having the focal point at the depth “Z_{Fm}”.

Where two transmission focal point positions “F₁” and “F₂” are set, the obtained reception signals are represented with “IQ (x,z;F₁)” and “IQ (x,z;F₂)”. In the scan sequence that walks the raster, if the transmission focal point is changed from “F₁” to “F₂”, the transmission beam is changed from “Tₖ” to “Tₖ₊₁”. Accordingly, the correction unit 132 multiplies “IQ (x,z;F₁)” by “W_{Fm} (x(n),z;Tₖ)” and “IQ (x,z;F₂)” by “W_{Fm} (x(n),z;Tₖ₊₁)”.

The signals the combining unit 133 outputs through addition and composition are represented by the following Expression (2).

IQ(x,z)=W_{F}_{₁}(x,z;Tₖ)·IQ(x,z;F₁)+W_{F}_{₂}(x,z;Fₖ₊₁)·IQ(x,z;F₂) (2)

It should be noted that, in the scan sequence in the above-described modification, if the transmission focal point is changed from “F₁” to “F₂”, the transmission beam remains “Tₖ”. In this example, the correction unit 132 multiplies “IQ (x,z;F₁)” by “W_{Fm}(x(n),z;Tₖ)” and “IQ (x,z;F₂)” by “W_{Fm}(x(n),z;Tₖ)”. The signals the combining unit 133 outputs through addition and composition are represented by the following Expression (3).

IQ(x,z)=W_{F}_{₁}(x,z;Tₖ)·IQ(x,z;F₁)+W_{F}_{₂}(x,z;Tₖ)·IQ(x,z;F₂) (3)

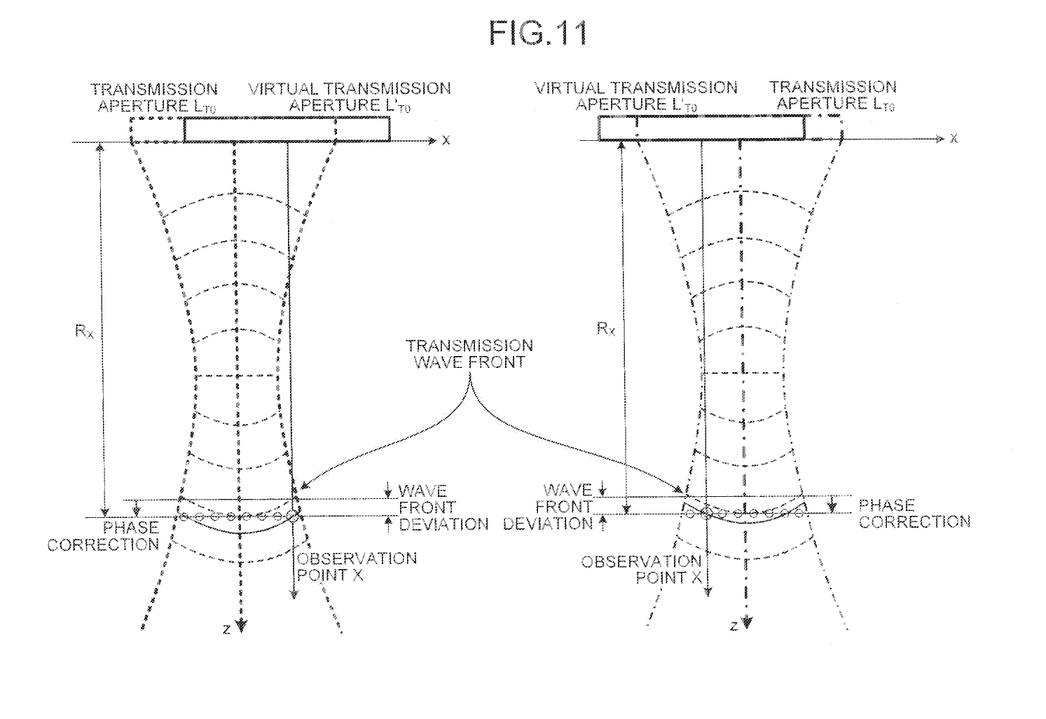

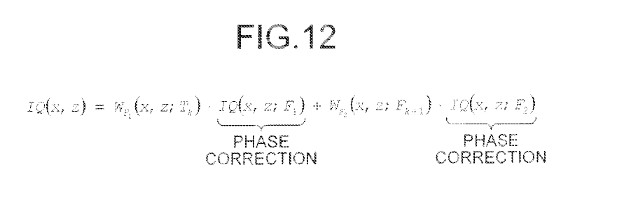

Expressions (2) and (3) represent the composite process after executing the weighting process of the amplitude only. Actually, a delay correction (a phase correction) described below is additionally executed by the correction unit 132. The phase correction will now be described with reference to FIGS. 11 FIG. 12. It should be noted that FIG. 11 illustrates that the transmission focal points on the same position are used for the right-hand and left-hand transmission beams, for drawing convenience. The transmission focal point positions, however, may be different on the right-hand and left-hand transmission beams, in the same manner as illustrated in FIG. 5.

FIG. 11 illustrates eight sample points simultaneously received at the depth “Rₓ” set on the M-th transmission beam and other eight sample points simultaneously received at the depth “Rₓ” set on the (M+1)-th transmission beam. FIG. 11 also illustrates that the right-end two points out of the eight sample points set on the M-th transmission beam are on the same position as the left-end two points out of the eight sample points set on the (M+1)-th transmission beam because of the number of overlaps setting. In addition, FIG. 11 illustrates the observation point X for both the right-most end point out of the eight sample points set on the M-th transmission beam and the second point from the left-end out of the eight sample points set on the (M+1)-th transmission beam.

Increased number of simultaneous reception points increases the gaps between the arrival times of the wave front of the transmission beam, in particular on the reception points on both ends. That is, as illustrated in FIG. 11, wave front deviation (phase deviation) is present between the phase of the transmission wave front that arrives at the observation point X apart from the transmission scanning line and the phase of the transmission wave front that arrives at a point in the vicinity of the transmission scanning line, although they are at the same depth. In addition, it is apparent by comparing the right-hand and left-hand diagrams on FIG. 11 with each other that the degree of the wave front deviation (phase deviation) varies depending on the position of the transmission scanning line although the identical observation point X is used. Accordingly, if the two reception signals at the observation point X are combined with each other without any transmission delay correction, the resultant signals are intensified or diminished with each other depending on the consistency and inconsistency of the phase. To cope with this, relative delay amount is corrected resulting from the differences in the propagation distances in transmission, reception, or transmission and reception.

The correction unit 132 then corrects the relative delay amount resulting from the differences in the propagation distances in transmission together with the above-described amplitude weighting process. The correction unit 132 calculates, for example, the arrival time of the transmission beam from “the transmission aperture L_{TO}” to reach the depth “R_{X}” on the transmission scanning line. The correction unit 132 sets “the virtual transmission aperture L′_{T0}” for forming a transmission beam on the virtual transmission scanning line passing through the observation point X and calculates the arrival time of the transmission beam from “the virtual transmission aperture L′_{T0}” to reach the observation point X. Subsequently, the correction unit 132 converts the time difference between these arrival times into the phase difference to calculate the phase correction amount. The above-described process is applied in the same manner to the transmission beams on the right-hand and left-hand diagrams illustrated in FIG. 11.

As illustrated in FIG. 12, the correction unit 132 executes, for example, the phase correction on “IQ (x,z;F₁)” in Expression (2) based on the arrival time difference of the transmission wave front, and the phase correction on “IQ (x,z;F₂)” in Expression (2) based on the arrival time difference of the transmission wave front. The combining unit 133 combines the corrected reception signals that are the processed reception signals subject to the amplitude correction and the phase correction executed by the correction unit 132, for each reception scanning line, then generates signals for one frame, and outputs the signals to the B-mode processing unit 14.

In the description above, “the distance from the center of the transmission beam to the reception scanning line” representing the reception scanning line position and the positional relation between the transmission beams, and, “the transmission beam width geometrically calculated” are used to calculate the weighting function based on the Gaussian distribution, and the amplitude weighting process (the amplitude correction) is executed. The present embodiment is not limited, however, to the above-described example. For another example, the correction unit 132 may simply determine the ratio between the distance from the center of the transmission beam and the transmission beam width as the weighting function.

Still another example, the correction unit 132 may use the sound field intensity on the reception scanning lines of the transmission ultrasonic wave rather than a geometrical parameter as the parameter in regard to the transmission ultrasonic wave. The correction unit 132, for example, may measure the sound field distribution of the transmission beam using a hydrophone without assuming the sound field intensity of the transmission beam as the Gaussian distribution, and calculate the weighting function based on the measured sound field intensity.

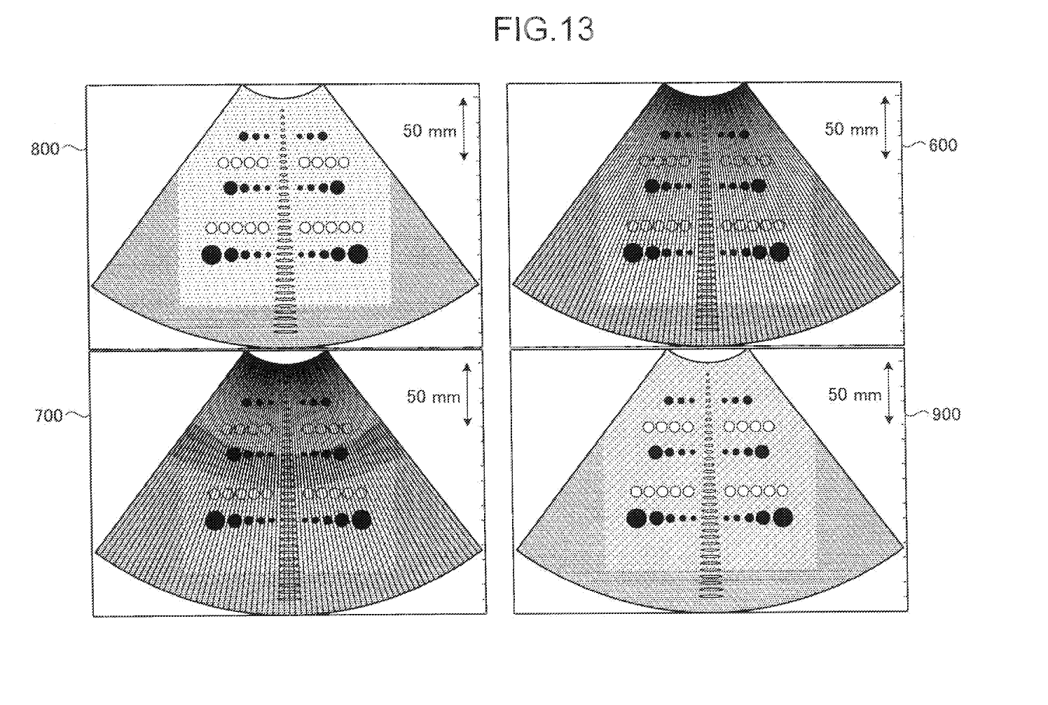

An image data 800 illustrated in FIG. 13 schematically illustrates a piece of B-mode image data obtained through the above-described amplitude weighting process and phase correction. The image data 800 is obtained through the scan sequence illustrated in FIG. 5 with the setting “F₁=80 mm, F₂=40 mm” and the parallel and simultaneous reception under the setting “the number of simultaneous receptions: 8, the number of overlaps: 2”. The image data 600 illustrated in FIG. 13 corresponds to the image data 600 illustrated in FIG. 3. The image data 600 here is the resultant data to which the transmission wave front composition is applied in addition to the conventional parallel and simultaneous reception under the setting “the number of simultaneous receptions: 8, the number of overlaps: 2”. The image data 700 illustrated in FIG. 13 corresponds to the image data 700 illustrated in FIG. 3. The image data 700 here is the resultant data to which the multi-stage focus is applied in addition to the conventional parallel and simultaneous reception under the setting “the number of simultaneous receptions: 8, the number of overlaps: 2”. An image data 900 illustrated in FIG. 13 schematically illustrates a piece of B-mode image data obtained through the typical B-mode scanning without executing the parallel and simultaneous reception.

As illustrated in FIG. 13, in the image data 800, “the stripes at intervals of the simultaneous reception” or “the irregularities of addition depending on presence of the addition and composition” disappear, which are found in the image data 600 and 700. The image quality of the image data 800 and the image data 900 are almost the same as illustrated in FIG. 13. Because the setting is “the number of simultaneous receptions: 8, the number of overlaps: 2”, the frame rate obtained by the image data 800 is six times as large as the frame rate obtained in the image data 800 in usual scanning.

In the above-described embodiment, both the time resolution and the spatial resolution are increased through executing, for example, the scan sequence described with reference to FIG. 5, the reception phasing addition by the receiver 12, the amplitude correction and the transmission delay correction (the phase correction of the transmission wave front) by the transmission phasing unit 13.

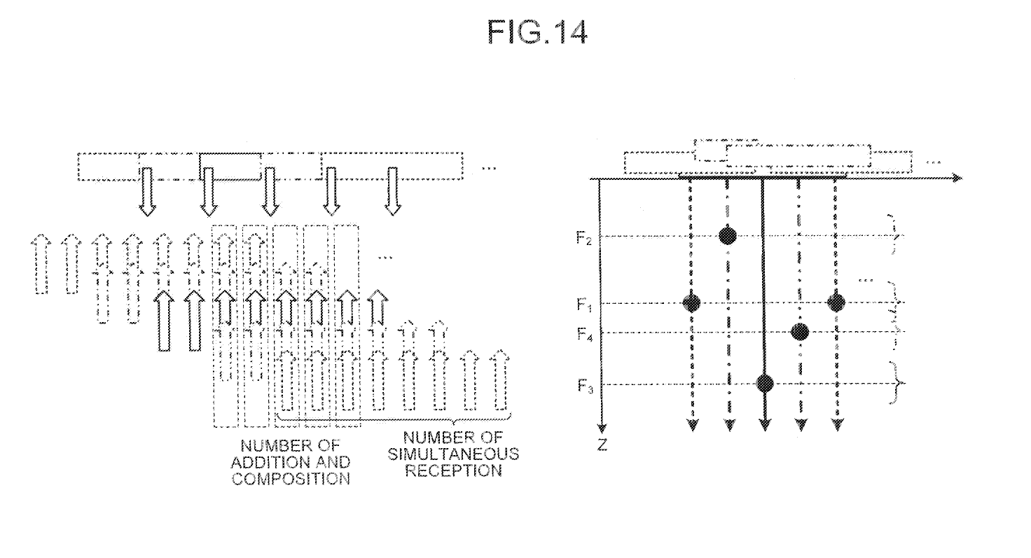

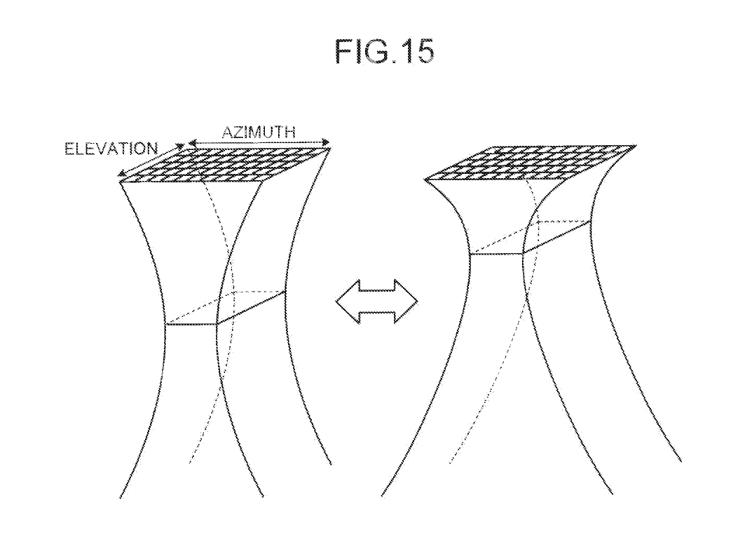

The following modifications may be made to the above-described embodiment. The following describes some modifications according to the present embodiment with reference to FIGS. 14 and 15. FIG. 14 is a diagram for explaining a first modification according to the present embodiment. FIG. 15 is a diagram for explaining a second modification according to the present embodiment.

The first modification will now be described. In the above-described embodiment, for the purpose of increasing the time resolution (the frame rate) as high as possible, the number of overlaps is set as small as possible to the extent not usually set so as to prevent the irregularities of addition from being generated. If such a high time resolution is not necessarily required, the controller 19 may set the number of overlaps (the number of addition and composition) larger under the following constraints. Specifically, as the first modification, the controller 19 determines the number of transmission focal points equal to the number of processed reception signals (the number of corrected reception signals) subject to the composition process executed by the combining unit 133 on a scanning line.

The left-hand diagram on FIG. 14 illustrates that the scan sequence is set to “the number of simultaneous receptions: 8, the number of overlaps: 6” so as to double the frame rate obtained in the usual scanning. The right-hand diagram on FIG. 14 illustrates that the scan sequence is set such that four transmission focal points “F₂, F₁, F₄, F₃” are set in this order from the smaller depth, the transmission focal point is switched in the order of “F₁, F₂, F₃, F₄” while shifting the transmission scanning line for each transmission rate.

In the scan sequence illustrated in FIG. 14, the smaller intervals of the simultaneous reception hardly generate the stripes resulting from the simultaneous reception. In addition, the number of transmission focal points is set equal to the number of corrected reception signals subject to the composition process, whereby as illustrated in FIG. 14, the number of addition and composition is always set to “4” on all of the reception sample points excluding the end portions within the scanning zone. Therefore, the irregularities of addition resulting from the difference of the number of addition and composition do not occur.

In the scan sequence illustrated in FIG. 14, the number of transmission focal points set equal to the number of addition and composition generates effects of the multi-stage focus, thereby obtaining uniform transmission beams. In addition, in the scan sequence illustrated in FIG. 14, the effects of the transmission wave front composition increase the spatial resolution, even if the position is away from the transmission focal point.

Furthermore, also in the first modification in which “the stripes resulting from the parallel and simultaneous reception” and “the irregularities of addition” hardly occur, the correction unit 132 executes the amplitude weighting process and the phase correction, thereby still increasing the spatial resolution.

The second modification will now be described. In the above-described embodiment, a plurality of transmission focal points are set when a 1-D array probe is used as the ultrasonic probe 1 to execute two-dimensional scanning. The ultrasonic wave imaging method according to the embodiment described above may be applied to three-dimensional scanning using a mechanical 4-D probe or a 2-D array probe as the ultrasonic probe 1. When the mechanical 4-D probe is used as the ultrasonic probe 1, for example, a piece of volume data is generated by combining a plurality of tomographic images obtained by mechanically swinging the elements. In this example, the ultrasonic wave imaging method according to the embodiment described above is executed on the tomographic images, thereby increasing both the time resolution and the spatial resolution.

By contrast, if the ultrasonic probe 1 is a 2-D array probe having a plurality of elements aligned in two dimensions, a plurality of transmission focal points may be set by driving the elements in one of the two alignment directions or by driving the elements in both the two alignment directions.

As illustrated in FIG. 15, the transmitter 11 transmits a transmission beam having a transmission focal point focused in the azimuth direction from a two-dimensional transmission aperture while switching the depth position of the transmission focal point for each transmission rate, for example. For another example, the transmitter 11 transmits a transmission beam having a transmission focal point focused in the elevation direction from a two-dimensional transmission aperture while switching the depth position of the transmission focal point for each transmission rate. For still another example, the transmitter 11 transmits a transmission beam having a transmission focal point focused in both the azimuth direction and the elevation direction from a two-dimensional transmission aperture while switching the depth position of the transmission focal point for each transmission rate.

The above-described transmission focal point control achieves a scan sequence similar to the scan sequence illustrated in FIG. 5, also in the three-dimensional scanning using a 2-D array probe. In the second modification, therefore, both the time resolution and the spatial resolution can be increased even if a piece of volume data is photographed. The second modification can be applied to the scanning using a 1.5-D array probe having the smaller number of elements in the elevation direction than that of the 2-D array probe.

### Control by Changing the Sound Field

The processes in the above-described embodiment can also be achieved without changing the transmission focal point if the shape of the transmission beam (refer to FIG. 9), that is, the sound field is changed.

For example, the transmitter 11 changes the width of the transmission aperture for transmitting the transmission ultrasonic wave for each transmission ultrasonic wave, thereby changing the sound field. This operation achieves the various types of processes described in the embodiment regardless of whether changing the transmission focal point. In addition, the various types of processes described in the embodiment can be achieved if the transmitter 11 changes the sound field by changing the width of the transmission aperture and the transmission focal point.

That is, in the ultrasonic diagnostic apparatus according to the above-described embodiment, the receiver 12 outputs a plurality of reception signals corresponding to the respective reception scanning lines for each transmission and reception of the ultrasonic wave by the ultrasonic probe 1. The correction unit 132 as a processor executes the weighting process and the phase correction process based on the reception scanning line position on at least one of the reception signals and a plurality of signals based on the reception signals, and outputs the processed signals for each reception scanning line. The combining unit 133 outputs a plurality of composite signals using a plurality of processed signals output by the correction unit 132 based on the transmission and reception of the ultrasonic waves before and after changing the sound field of the transmitted ultrasonic waves, and before and after changing the position of a plurality of reception scanning lines. Subsequently, the image generator 16 generates a piece of image data based on a plurality of composite signals output by the combining unit 133. This operation enables the ultrasonic diagnostic apparatus according to the embodiment to increase both the time resolution and the spatial resolution.

For example, the transmitter 11 changes the sound field every predetermined number of times of transmission and reception of the ultrasonic wave by the ultrasonic probe 1. Specifically, the transmitter 11 changes at least one of the position of the transmission focal point and the width of the transmission aperture for each transmission ultrasonic wave, thereby changing the sound field. Subsequently, the controller 19 changes a plurality of reception scanning line positions every predetermined number of times of transmission and reception of the ultrasonic wave by the ultrasonic probe 1. This operation enables the ultrasonic diagnostic apparatus according to the embodiment to generate an image having reduced stripes and irregularities of addition by utilizing the difference of the sound fields (i.e., the transmission beams with different shapes).

The processes in the above-described embodiment can also be applied to the scanning in which the sound field is changed by changing the width of the transmission aperture.

### Weighting Process

In the description above, the weighting process and the phase correction process are executed; however, the present embodiment is not limited to these examples. For another example, a weighting process without the phase correction process also results in similar advantageous effects. Specifically, the parallel and simultaneous reception can be executed with an arbitrarily set number of overlaps by executing the weighting process without the phase correction process.

The following describes this with reference to some examples. In the above-described embodiment, the parallel and simultaneous reception executed under the setting “the number of simultaneous receptions: 8, the number of overlaps: 2” is executed by executing the weighting process. The description is provided merely for exemplary purpose and not limiting. In the parallel and simultaneous reception under the setting of the number of simultaneous receptions “8”, for example, the number of overlaps may be set to either one of the values “3”, “5”, “6”, and “7”.

For example, the parallel and simultaneous reception under the setting “the number of simultaneous receptions: 8, the number of overlaps: 3” is executed. In the parallel and simultaneous reception, if the reception scanning line position is changed from the M-th transmission beam to the (M+1)-th transmission beam, three reception scanning lines are on the same positions before and after the change, and five reception scanning lines are on different positions before and after the change, out of the reception scanning lines in the M-th transmission beam (eight lines) and the reception scanning lines in the (M+1)-th transmission beam (eight lines).

For another example, if the parallel and simultaneous reception under the setting “the number of simultaneous receptions: 8, the number of overlaps: 5” is executed, five reception scanning lines are on the same positions before and after the change, and three reception scanning lines are on different positions before and after the change, out of the reception scanning lines in the M-th transmission beam and the reception scanning lines in the (M+1)-th transmission beam.

As described above, the weighting process achieves the parallel and simultaneous reception using any integer from one up to the number of simultaneous receptions (eight in this example) as the number of overlaps.

That is, in the ultrasonic diagnostic apparatus according to the above-described embodiment, the receiver 12 outputs a plurality of reception signals corresponding to the respective reception scanning lines for each transmission and reception of the ultrasonic wave by the ultrasonic probe. The correction unit 132 as a processor executes the weighting process based on the reception scanning line position on a plurality of reception signals or a plurality of signals based on a plurality of reception signals, and outputs the processed signals for each reception scanning line. The combining unit 133 outputs a plurality of composite signals using a plurality of signals including a plurality of processed signals output by the processor based on at least the transmission and reception of the ultrasonic waves before and after changing the position of a plurality of reception scanning lines. Subsequently, the image generator 16 generates a piece of image data based on a plurality of composite signals output by the combining unit 133. The position of a plurality of reception scanning lines is changed such that the position of the number, other than divisors of the reception scanning lines, of reception scanning lines is different before and after the change, and the position of the remaining number (the number of overlaps) of the reception scanning lines is the same before and after the change. This operation enables the ultrasonic diagnostic apparatus according to the embodiment to increase the signal-to-noise ratio, reduce the irregularities of addition and the stripes, and furthermore, increase the frame rate.

Also in the above-described embodiment, any number may be set as the number of overlaps. It should be noted that the number of simultaneous receptions available for the parallel and simultaneous reception is not limited to “8” and another number may be set.

Other Configurations

The respective components in the respective apparatuses shown in the explanation of the first to the second embodiments are of functional concept, and it is not necessarily required to be physically configured as shown in the drawings. Specifically, a specific form of distribution and integration of the respective devices are not limited to the ones shown in the drawings, and it can be configured such that all or a part thereof is functionally or physically distributed or integrated in arbitrary units according to various kinds of load and usage condition and the like. Furthermore, as for the respective processing functions of the respective devices, all or an arbitrary part thereof can be implemented by a central processing unit (CPU) and a computer program that is analyzed and executed by the CPU, or can be implemented as hardware by wired logic.

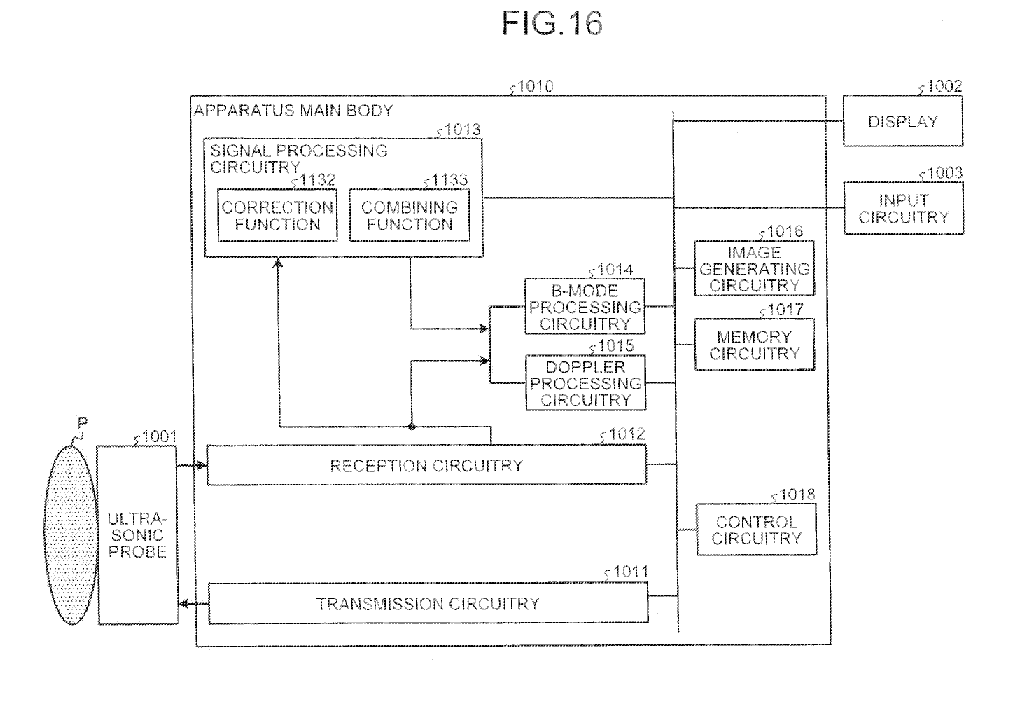

For example, the ultrasonic diagnostic apparatus shown in FIG. 1 may be configured as shown in FIG. 16. FIG. 16 is a block diagram illustrating the configuration of an ultrasonic diagnostic apparatus according to other embodiments.

As illustrated in FIG. 16, an ultrasonic diagnostic apparatus includes an ultrasonic probe 1001, a display 1002, input circuitry 1003, and an apparatus main body 1010. The ultrasonic probe 1001, the display 1002, the input circuitry 1003, and the apparatus main body 1010 correspond to the ultrasonic probe 1, the monitor 2, the input device 3, and the apparatus main body 10 shown in FIG. 1, respectively.

The apparatus main body 1010 includes transmission circuitry 1011, reception circuitry 1012, signal processing circuitry 1013, B-mode processing circuitry 1014, Doppler processing circuitry 1015, image generating circuitry 1016, memory circuitry 1017, and control circuitry 1018. The transmission circuitry 1011, the reception circuitry 1012, the signal processing circuitry 1013, the B-mode processing circuitry 1014, the Doppler processing circuitry 1015, the image generating circuitry 1016, and the control circuitry 1018 correspond to the transmitter 11, the receiver 12, the transmission phasing unit 13, the B-mode processing unit 14, the Doppler processing unit 15, the image generator 16, and the controller 19 shown in FIG. 1, respectively. The memory circuitry 1017 correspond to the image memory 17 and the internal storage unit 18 shown in FIG. 1. The signal processing circuitry 1013 is an example of signal processing circuitry in the accompanying claims. The image generating circuitry 1016 is an example of image generating circuitry in the accompanying claims.

The signal processing circuitry 1013 performs a correction function 1132 and a combining function 1133. The correction function 1132 is a function implemented by the correction unit 132 illustrated in FIG. 1. The combining function 1133 is a function implemented by the combining unit 133 illustrated in FIG. 1.

For example, each of the respective processing functions performed by the correction function 1132 and the combining function 1133 which are components of the signal processing circuitry 1013 illustrated in FIG. 16, is stored in the memory circuitry 1017 in a form of a computer-executable program. The signal processing circuitry 1013 is a processor that loads programs from the memory circuitry 1017 and executes the programs so as to implement the respective functions corresponding to the programs. In other words, the signal processing circuitry 1013 that has loaded the programs has the functions illustrated in the signal processing circuitry 1013 in FIG. 16. That is, the signal processing circuitry 1013 loads a program corresponding to the correction function 1132 from the memory circuitry 1017 and executes the program so as to perform the same processing as that of the correction unit 132. The signal processing circuitry 1013 loads a program corresponding to the combining function 1133 from the memory circuitry 1017 and executes the program so as to perform the same processing as that of the combining unit 133.

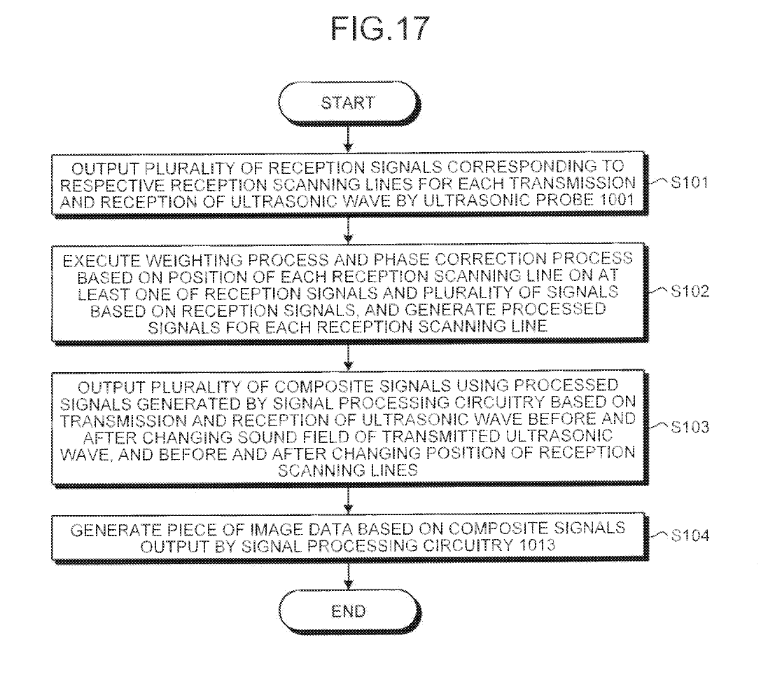

FIG. 17 is a flowchart of a processing procedure of the ultrasonic diagnostic apparatus according to other embodiments. As shown in FIG. 17, the reception circuitry 1012 outputs a plurality of reception signals corresponding to respective reception scanning lines for each transmission and reception of an ultrasonic wave by an ultrasonic probe (step S101). The signal processing circuitry 1013 executes a weighting process and a phase correction process based on a position of each reception scanning line on at least one of the reception signals and a plurality of signals based on the reception signals, and generates processed signals for each reception scanning line (step S102). The signal processing circuitry 1013 outputs a plurality of composite signals using the processed signals generated by the signal processing circuitry based on the transmission and reception of the ultrasonic wave before and after changing a sound field of a transmitted ultrasonic wave, and before and after changing the position of the reception scanning lines (step S103). The image generating circuitry 1016 generates a piece of image data based on the composite signals output by the signal processing circuitry (step S104).

For example, Steps S102 illustrated in FIG. 17 is a step that is implemented by the signal processing circuitry 1013 loading the program corresponding to the correction function 1132 from the memory circuitry 1017 and executing the program. Step S103 illustrated in FIG. 17 is a step that is implemented by the signal processing circuitry 1013 loading the program corresponding to the combining function 1133 from the memory circuitry 1017 and executing the program.

In FIG. 16, the processing functions performed by the correction function 1132 and the combining function 1133 are described as being implemented in the single processing circuit (signal processing circuitry). The functions, however, may be implemented by configuring a processing circuit by combining a plurality of separate processors and causing each of the processors to execute a program.

The term “processor” used in the above description means, for example, a central preprocess unit (CPU) and a graphics processing unit (GPU), or a circuit such as an application specific integrated circuit (ASIC), a programmable logic device (for example, a simple programmable logic device (SPLD)), a complex programmable logic device (CPLD), and a field programmable gate array (FPGA). The processor implements a function by loading and executing a program stored in a storage circuit. Instead of being stored in a storage circuit, the program may be built directly in a circuit of the processor. In this case, the processor implements a function by loading and executing the program built in the circuit. The processors in the present embodiment are not limited to a case in which each of the processors is configured as a single circuit. A plurality of separate circuits may be combined as one processor that implements the respective functions. Furthermore, the components illustrated in FIG. 16 may be integrated into one processor that implements the respective functions.

The respective circuitry exemplified in FIG. 16 may be distributed or integrated as appropriate. For example, the signal processing circuitry 1013 and the control circuitry 1018 may be integrated.

It should also be noted that the ultrasonic wave imaging method described in the above-described embodiment and modifications may be provided separate from the ultrasonic diagnostic apparatus. That is, a signal processing device having the function of the above-described transmission phasing unit 13 and the like may obtain the reception signals from the receiver 12 and execute processes.

Among those processes explained in the embodiment and modifications, the whole or a part of the processes explained to be executed automatically may also be executed manually. Furthermore, the whole or a part of the processes explained to be performed manually may be performed automatically by known methods. In addition, processing or controlling procedures, specific names, information including various types of data and parameters may be modified in any manner unless specified otherwise.

Furthermore, the devices illustrated in the drawings are merely a depiction of concepts or functionality, and is not necessarily configured physically in the manner illustrated in the drawings. In other words, specific configurations in which the devices are divided or integrated are not limited to those illustrated in the drawings. More specifically, the whole or a part of the devices may be divided or integrated functionally or physically in any units depending on various loads or utilization. For example, the transmission phasing unit 13 illustrated in FIG. 1 may be integrated into the receiver 12. The whole or a part of the processing functions executed in each of the devices may be implemented as a CPU and a computer program parsed and executed by the CPU, or implemented as hardware using wired logics.

The ultrasonic wave imaging method explained in the embodiment and modifications can be implemented by causing a computer, such as a personal computer or a workstation, to execute an ultrasonic wave imaging program prepared in advance. The ultrasonic wave imaging method may be distributed over a network such as the Internet. Furthermore, the ultrasonic wave image processing method may also be provided in a manner recorded in a computer-readable recording medium, such as a hard disk, a flexible disk (FD), a compact disc read-only memory (CD-ROM), a magneto-optical disk (MO), and a digital versatile disc (DVD), and be executed by causing a computer to read the method from the recording medium.

As described above, according to the above-described embodiment and modifications, both the time resolution and the spatial resolution can be increased.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
reception circuitry configured to output a plurality of reception signals corresponding to respective reception scanning lines for each transmission and reception of an ultrasonic wave by an ultrasonic probe;
signal processing circuitry configured to
execute a weighting process and a phase correction process based on a position of each reception scanning line on at least one of the reception signals and a plurality of signals based on the reception signals, and generate processed signals for each reception scanning line, and
output a plurality of composite signals using the processed signals generated based on the transmission and reception of the ultrasonic wave before and after changing a sound field of a transmitted ultrasonic wave, and before and after changing the position of the reception scanning lines; and
image generating circuitry configured to generate a piece of image data based on the composite signals output by the signal processing circuitry.

2. An ultrasonic diagnostic apparatus comprising:
reception circuitry configured to output a plurality of reception signals corresponding to respective reception scanning lines for each transmission and reception of an ultrasonic wave by an ultrasonic probe;
signal processing circuitry configured to
execute a weighting process based on a position of each reception scanning line on at least one of the reception signals and a plurality of signals based on the reception signals, and generate processed signals for each reception scanning line, and
output a plurality of composite signals using a plurality of signals including the processed signals generated at least based on the transmission and reception of the ultrasonic wave before and after changing the position of the reception scanning lines; and
image generating circuitry configured to generate a piece of image data based on the composite signals output by the signal processing circuitry, wherein
the reception scanning line positions are changed such that the position of number, other than divisors of the reception scanning lines, of reception scanning lines is different before and after the change, and the position of a remaining number of the reception scanning lines is the same before and after the change.

3. The ultrasonic diagnostic apparatus according to claim 1, wherein the reception scanning line positions are changed such that the position of number, other than divisors of the reception scanning lines, of reception scanning lines is different before and after the change, and the position of a remaining number of the reception scanning lines is the same before and after the change.

4. The ultrasonic diagnostic apparatus according to claim 1, further comprising:
transmitting circuitry configured to change the sound field every predetermined number of times of transmission and reception of the ultrasonic wave by the ultrasonic probe; and
control circuitry configured to change the reception scanning line positions for every predetermined number of times of transmission and reception of the ultrasonic wave by the ultrasonic probe.

5. The ultrasonic diagnostic apparatus according to claim 2, further comprising:
transmitting circuitry configured to change a sound field of transmitted ultrasonic waves every predetermined number of times of transmission and reception of the ultrasonic wave by the ultrasonic probe; and
control circuitry configured to change the reception scanning line positions for every predetermined number of times of transmission and reception of the ultrasonic wave by the ultrasonic probe.

6. The ultrasonic diagnostic apparatus according to claim 1, wherein the signal processing circuitry is configured to output the composite signals by composing the processed signals relating to a common reception scanning line with each other.

7. The ultrasonic diagnostic apparatus according to claim 2, wherein the signal processing circuitry is configured to output the composite signals by composing the processed signals relating to a common reception scanning line with each other.

8. The ultrasonic diagnostic apparatus according to claim 4, wherein the transmitting circuitry is configured to change the sound field by changing at least one of a position of a transmission focal point of a transmission ultrasonic wave transmitted by the ultrasonic probe and a width of a transmission aperture configured to transmit the transmission ultrasonic wave, for each of the transmission ultrasonic waves.

9. The ultrasonic diagnostic apparatus according to claim 5, wherein the transmitting circuitry is configured to change the sound field by changing at least one of a position of a transmission focal point of a transmission ultrasonic wave transmitted by the ultrasonic probe and a width of a transmission aperture configured to transmit the transmission ultrasonic wave, for each of the transmission ultrasonic waves.

10. The ultrasonic diagnostic apparatus according to claim 8, wherein the transmitting circuitry is configured to change at least one of the position of the transmission focal point on a common transmission scanning line and the width of the transmission aperture, for each transmission ultrasonic wave.

11. The ultrasonic diagnostic apparatus according to claim 9, wherein the transmitting circuitry is configured to change at least one of the position of the transmission focal point on a common transmission scanning line and the width of the transmission aperture, for each transmission ultrasonic wave.

12. The ultrasonic diagnostic apparatus according to claim 8, wherein the signal processing circuitry is configured to calculate a weight of amplitude used for the weighting process and a phase correction amount used for the phase correction process on the reception signals based on the transmission focal point position of the transmission ultrasonic wave from which the reception signals are obtained.

13. The ultrasonic diagnostic apparatus according to claim 12, wherein the signal processing circuitry is configured to calculate the weight of amplitude on the reception signals based on a parameter in regard to the transmission ultrasonic wave from which the reception signals are obtained.

14. The ultrasonic diagnostic apparatus according to claim 13, wherein the signal processing circuitry is configured to use a distance from the transmission ultrasonic wave to the reception scanning lines, as the parameter in regard to the transmission ultrasonic wave.

15. The ultrasonic diagnostic apparatus according to claim 13, wherein the signal processing circuitry is configured to use a sound field intensity on each reception scanning line of the transmission ultrasonic wave as the parameter in regard to the transmission ultrasonic wave.

16. The ultrasonic diagnostic apparatus according to claim 12, wherein the signal processing circuitry is configured to calculate the phase correction amount on the reception signals based on relative differences in propagation paths for a transmission ultrasonic wave from which the reception signals are obtained to reach the respective reception scanning lines.

17. The ultrasonic diagnostic apparatus according to claim 9, wherein the transmitting circuitry is configured to determine the aperture width for transmitting a transmission ultrasonic wave for the transmission focal points to be any desirable aperture width, a fixed aperture width, or an aperture width depending on the transmission focal point position.

18. The ultrasonic diagnostic apparatus according to claim 9, wherein number of transmission focal points is equal to number of processed reception signals subject to the composite processing executed by the signal processing circuitry on a single scanning line.

19. A control method comprising:
outputting a plurality of reception signals corresponding to respective reception scanning lines for each transmission and reception of an ultrasonic wave by an ultrasonic probe;
executing a weighting process and a phase correction process based on a position of each reception scanning line on at least one of the reception signals and a plurality of signals based on the reception signals, and generate processed signals for each reception scanning line;
outputting a plurality of composite signals using the processed signals generated based on the transmission and reception of the ultrasonic wave before and after changing a sound field of a transmitted ultrasonic wave, and before and after changing the position of the reception scanning lines; and
generating a piece of image data based on the composite signals output by the signal processing circuitry.

20. An ultrasonic diagnostic apparatus comprising:
outputting a plurality of reception signals corresponding to respective reception scanning lines for each transmission and reception of an ultrasonic wave by an ultrasonic probe;
executing a weighting process based on a position of each reception scanning line on at least one of the reception signals and a plurality of signals based on the reception signals, and generate processed signals for each reception scanning line;
outputting a plurality of composite signals using a plurality of signals including the processed signals generated at least based on the transmission and reception of the ultrasonic wave before and after changing the position of the reception scanning lines; and
generating a piece of image data based on the composite signals output by the signal processing circuitry, wherein
the reception scanning line positions are changed such that the position of number, other than divisors of the reception scanning lines, of reception scanning lines is different before and after the change, and the position of a remaining number of the reception scanning lines is the same before and after the change.
